(19) United States
(12) Patent Application Publication
(10) Pub. No.: US 2020/0230304 A1
(43) Pub. Date: Jul. 23, 2020

(54) Collapsible Fluid Reservoir with Filter

(71) Applicant: Smisson-Cartledge Biomedical LLC, Macon, GA (US)

(72) Inventors: David C. Field, Eatonton, GA (US); Hugh F. Smisson, III, Macon, GA (US); Billy D. William, Stone Mountain, GA (US); Barry J. Feldman, Santa Cruz, CA (US)

(21) Appl. No.: 16/632,149

(22) PCT Filed: Jul. 6, 2018
(86) PCT No.: PCT/US2018/040994

§ 371 Date: Jan. 17, 2020

Related U.S. Application Data
(60) Provisional application No. 62/529,149, filed on Jul. 6, 2017.

(51) Int. Cl. A61M 1/02; A61J 1/10; A61J 1/14

(52) U.S. Cl. CPC A61M 1/0222; A61J 1/1475; A61J 1/1456; A61J 1/10

(57) ABSTRACT

A collapsible fluid reservoir may include a flexible container, a flexible filter, a first inflow line, a second inflow line, and an outflow line. The container may include a first sheet and a second sheet defining an internal space therebetween for containing a biological fluid. The filter may be disposed within the internal space and attached to the container along an entire periphery of the filter. The filter may include a first side facing the first sheet and a second side facing the second sheet. The first inflow line and the second inflow line each may be disposed at least partially within the interior space and between the first side of the filter and the first sheet. The outflow line may be disposed at least partially within the interior space and between the second side of the filter and the second sheet.

### CROSS-REFERENCE TO RELATED APPLICATIONS

This applications claims priority benefit to U.S. Provisional Application No. 62/529,149, filed Jul. 6, 2017, the entire contents of which are incorporated by reference herein.

### FIELD OF THE DISCLOSURE

The present disclosure relates generally to fluid reservoirs, and more particularly to collapsible fluid reservoirs having an integral filter and related methods for using such reservoirs to contain, filter, and transfer biological fluids, such as blood.

### BACKGROUND OF THE DISCLOSURE

Various types of fluid reservoirs may be used in medical procedures for collecting, storing, and transferring biological fluids. For example, during certain cardiologic procedures, one or more fluid reservoirs may be used as a part of an extracorporeal circuit to collect, contain, and/or deliver blood to a patient. Depending on the application, some fluid reservoirs may be rigid, while other fluid reservoirs may be collapsible such that the reservoir adjusts to the volume of fluid contained therein. In some instances, fluid reservoirs used in medical procedures may include a filter for removing undesired materials from the biological fluid. For example, fluid reservoirs used in the transfusion of blood may include a filter for removing clots and small clumps of platelets and white blood cells that may form during collection and storage of the blood.

Although existing fluid reservoirs generally may be suitable for use in certain medical procedures, such reservoirs may suffer from one or more potential drawbacks. For example, the throughput of certain fluid reservoirs may be limited by the configuration of an inlet portion and/or an outlet portion of the reservoir, which may extend the amount of time required to collect or transfer a biological fluid or may necessitate the use of additional reservoirs. Some fluid reservoirs may include a filter with a relatively small surface area contacting incoming biological fluid, which may extend the amount of time required to remove undesired materials. Additionally, certain fluid reservoirs having a filter may be configured in a manner that increases the likelihood of clot formation and/or clogging of the filter, which may necessitate replacement of the reservoir. Further, certain fluid reservoirs having a filter may lack a means for recirculating unfiltered biological fluid and/or a means for allowing biological fluid to bypass the filter, which may be desirable in some instances.

A need therefore exists for improved fluid reservoirs for use in medical procedures, which address one or more of the above-described potential drawbacks of existing technology and are able to be used in a broader range of applications for collecting, storing, and transferring biological fluids, such as blood.

### SUMMARY OF THE DISCLOSURE

Various embodiments described herein provide fluid reservoirs and related methods for use in medical procedures to collect, store, and transfer biological fluids. In certain embodiments, the fluid reservoirs may be collapsible and may include a flexible container and a flexible filter disposed within an interior space of the container. The filter may span the interior space and be attached to the container along an entire periphery of the filter. In this manner, the filter may divide the interior space into two portions, an inflow portion upstream of the filter, and an outflow portion downstream of the filter. Multiple inflow lines may be provided to deliver a biological fluid into the inflow portion of the interior space, and an outflow line may be provided to receive the filtered biological fluid from the outflow portion of the interior space. The fluid reservoirs may be used in an upright (i.e., vertical) orientation with the filter extending in a vertical manner and the inflow lines disposed above the outflow line.

According to one aspect, a collapsible fluid reservoir is provided for collecting, storing, and transferring a biological fluid. In one embodiment, a collapsible fluid reservoir may include a flexible container, a flexible filter, a first inflow line, a second inflow line, and an outflow line. The container may include a first sheet and a second sheet attached to one another and defining an internal space therebetween for containing a biological fluid. The filter may be disposed within the internal space and attached to the container along an entire periphery of the filter, and the filter may include a first side facing the first sheet and a second side opposite the first side and facing the second sheet. The first inflow line may be disposed at least partially within the interior space and between the first side of the filter and the first sheet. The second inflow line may be disposed at least partially within the interior space and between the first side of the filter and the first sheet. The outflow line may be disposed at least partially within the interior space and between the second side of the filter and the second sheet.

In certain embodiments, the first sheet and the second sheet may be attached to one another along the periphery of the filter. In certain embodiments, the first sheet and the second sheet may be attached to one another by a seal extending along the periphery of the filter. In certain embodiments, the first sheet and the second sheet may be formed of polyvinyl chloride. In certain embodiments, the filter may include a sheet of filter material. In certain embodiments, the filter may include a plurality of sheets of filter material. In certain embodiments, the filter may have a pore size between 1 micron and 1000 microns. In certain embodiments, the filter may have a pore size between 1 micron and 500 microns. In certain embodiments, the filter may have a pore size between 200 micron and 250 microns. In certain embodiments, the filter may have a pore size that is substantially constant throughout the filter. In certain embodiments, the filter may have a pore size that decreases in a direction from a first end to an opposite second end of the filter. In certain embodiments, the collapsible fluid reservoir may be configured for use in a vertical orientation with the first end of the filter disposed above the second end of the filter. In certain embodiments, the collapsible fluid reservoir may be configured for use in a vertical orientation with the second end of the filter disposed above the first end of the filter. In certain embodiments, the filter may include a first region having a first pore size and a second region having a second pore size, with the first pore size being greater than the second pore size. In certain embodiments, the filter also may include a third region having a third pore size, with the second pore size being greater than the third pore size. In certain embodiments, the first region and the second region of the filter may be integrally formed with one another. In certain embodiments, the first region and the second region of the filter may be separately formed and attached to one another. In certain embodiments, the filter may be attached to at least one of the first sheet and the second sheet along the entire periphery of the filter.

In certain embodiments, the first inflow line and the second inflow line may be disposed along a first end of the container, the outflow line may be disposed along an opposite second end of the container, and the collapsible fluid reservoir may be configured for use in a vertical orientation with the first end of the container disposed above the second end of the container. In certain embodiments, the first inflow line may include a first inflow port disposed at least partially within the interior space and a first inflow tube attached to the first inflow port and disposed at least partially outside of the interior space, and the second inflow line may include a second inflow port disposed at least partially within the interior space and a second inflow tube attached to the second inflow port and disposed at least partially outside of the interior space. In certain embodiments, the outflow line may include an outflow port disposed at least partially within the interior space and an outflow tube attached to the outflow port and disposed at least partially outside of the interior space.

In certain embodiments, the collapsible fluid reservoir also may include a third inflow line disposed at least partially within the interior space. In certain embodiments, the third inflow line may be disposed between the first side of the filter and the first sheet. In certain embodiments, the third inflow line may be disposed between the second side of the filter and the second sheet. In certain embodiments, the first inflow line, the second inflow line, and the third inflow line may be disposed along a first end of the container, the outflow line may be disposed along an opposite second end of the container, and the collapsible fluid reservoir may be configured for use in a vertical orientation with the first end of the container disposed above the second end of the container. In certain embodiments, the collapsible fluid reservoir also may include a return line disposed at least partially within the interior space and between the first side of the filter and the first sheet. In certain embodiments, the first inflow line and the second inflow line may be disposed along a first end of the container, the outflow line and the return line may be disposed along an opposite second end of the container, and the collapsible fluid reservoir may be configured for use in a vertical orientation with the first end of the container disposed above the second end of the container. In certain embodiments, the collapsible fluid reservoir also may include a vent line disposed at least partially within the interior space and between the first side of the filter and the first sheet. In certain embodiments, the first inflow line, the second inflow line, and the vent line may be disposed along a first end of the container, the outflow line may be disposed along an opposite second end of the container, and the collapsible fluid reservoir may be configured for use in a vertical orientation with the first end of the container disposed above the second end of the container.

In another embodiment, a collapsible fluid reservoir may include a flexible container, a flexible filter, a first inflow line, a second inflow line, and an outflow line. The container may include a first sheet and a second sheet attached to one another and defining an internal space therebetween for containing a biological fluid. The filter may be disposed within the internal space, and the filter may include a first side facing the first sheet and a second side opposite the first side and facing the second sheet. The first inflow line may be disposed at least partially within the interior space and between the first side of the filter and the first sheet. The second inflow line may be disposed at least partially within the interior space and between the second side of the filter and the second sheet. The outflow line may be disposed at least partially within the interior space and between the second side of the filter and the second sheet. In certain embodiments, the first inflow line and the second inflow line may be disposed along a first end of the container, the outflow line may be disposed along an opposite second end of the container, and the collapsible fluid reservoir may be configured for use in a vertical orientation with the first end of the container disposed above the second end of the container.

In still another embodiment, a collapsible fluid reservoir may include a flexible container, a flexible filter, an inflow line, an outflow line, and a return line. The container may include a first sheet and a second sheet attached to one another and defining an internal space therebetween for containing a biological fluid. The filter may be disposed within the internal space, and the filter may include a first side facing the first sheet and a second side opposite the first side and facing the second sheet. The inflow line may be disposed at least partially within the interior space and between the first side of the filter and the first sheet. The outflow line may be disposed at least partially within the interior space and between the second side of the filter and the second sheet. The return line may be disposed at least partially within the interior space and between the first side of the filter and the first sheet. In certain embodiments, the inflow line may be disposed along a first end of the container, the outflow line and the return line may be disposed along an opposite second end of the container, and the collapsible fluid reservoir may be configured for use in a vertical orientation with the first end of the container disposed above the second end of the container.

In another embodiment, a collapsible fluid reservoir may include a flexible container, a flexible filter, an inflow line, a first outflow line, and a second outflow line. The container may include a first sheet and a second sheet attached to one another and defining an internal space therebetween for containing a biological fluid. The filter may be disposed within the internal space, and the filter may include a first side facing the first sheet and a second side opposite the first side and facing the second sheet. The inflow line may be disposed at least partially within the interior space and between the first side of the filter and the first sheet. The first outflow line may be disposed at least partially within the interior space and between the second side of the filter and the second sheet. The second outflow line may be disposed at least partially within the interior space and between the first side of the filter and the first sheet. In certain embodiments, the inflow line may be disposed along a first end of the container, the first outflow line and the second outflow line may be disposed along an opposite second end of the container, and the collapsible fluid reservoir may be configured for use in a vertical orientation with the first end of the container disposed above the second end of the container.

In still another embodiment, a collapsible fluid reservoir may include a flexible container, a flexible filter bag, an inflow line, and an outflow line. The container may include a first container sheet and a second container sheet attached to one another and defining a first internal space therebetween for containing a biological fluid. The filter bag may be attached to the container and disposed at least partially within the internal first space. The filter bag may have a first side facing the first container sheet and a second side disposed opposite the first side and facing the second container sheet, and the filter bag may define a second internal space therein. The inflow line may be disposed at least partially within the second internal space and at least partially within the first internal space. The outflow line may be disposed outside of the second internal space and at least partially within the first internal space.

In certain embodiments, the first container sheet and the second container sheet may be attached to one another by a container seal extending along the outer periphery of the container, and the filter bag may be attached to the container by the container seal. In certain embodiments, the filter bag may have a first end, a second end disposed opposite the first end, a third side, and a fourth side disposed opposite the third side. The filter bag may be attached to the container by the container seal along the first end of the filter bag, and the filter bag may be unattached to the container along the second end, the first side, the second side, the third side, and the fourth side of the filter bag. In certain embodiments, the filter bag may include a first filter sheet and a second filter sheet attached to one another and defining the second internal space therebetween. In certain embodiments, the first filter sheet and the second filter sheet may be attached to one another by a filter seal extending along the second end, the third side, and the fourth side of the filter bag.

In certain embodiments, the collapsible fluid reservoir also may include a flexible second filter bag attached to the container and disposed at least partially within the second internal space and at least partially within the first internal space, and the second filter bag may define a third interior space therein. In certain embodiments, the second filter bag may have a first end and a second end disposed opposite the first end. The second filter bag may be attached to the container by the container seal along the first end of the second filter bag, and the second filter bag may be unattached to the container along the second end of the filter bag. In certain embodiments, the second filter bag may include a first filter sheet and a second filter sheet attached to one another by a filter seal and defining the third internal space therebetween. In certain embodiments, a pore size of the second filter bag may be less than a pore size of the first filter bag. In certain embodiments, a volume of the third interior space may be less than a volume of the second interior space. In certain embodiments, the inflow line may be disposed outside of the third internal space. In certain embodiments, the collapsible fluid reservoir also may include a second inflow line disposed at least partially within the third internal space, at least partially within the second internal space, and at least partially within the first internal space.

In certain embodiments, the collapsible fluid reservoir also may include a second inflow line, a vent line, and a return line. The second inflow line may be disposed at least partially within the second internal space and at least partially within the first internal space. The vent line may be disposed at least partially within the second internal space and at least partially within the first internal space. The return line disposed outside of the second internal space and at least partially within the first internal space. In certain embodiments, the inflow line may be disposed along a first end of the container, and the outflow line may be disposed along an opposite second end of the container. The collapsible fluid reservoir may be configured for use in a vertical orientation with the first end of the container disposed above the second end of the container.

These and other aspects and embodiments of the present disclosure will be apparent or will become apparent to one of ordinary skill in the art upon review of the following detailed description when taken in conjunction with the several drawings and the appended claims.

### BRIEF DESCRIPTION OF THE DRAWINGS

In describing the various embodiments of the present disclosure, reference is made to the accompanying drawings, which are not necessarily drawn to scale, and wherein:

FIG. 1A is a front view of a fluid reservoir in accordance with one or more embodiments of the present disclosure, showing a container, a number of inflow lines, a vent line, a number of auxiliary lines, an outflow line, and a return line of the fluid reservoir.

FIG. 1B is a detailed cross-sectional bottom view of the fluid reservoir of FIG. 1A, taken along line 1B-1B, showing the container, the inflow lines, the vent line, the auxiliary lines, and a filter and a defoamer of the fluid reservoir disposed within an interior space of the container.

FIG. 1C is a detailed cross-sectional top view of the fluid reservoir of FIG. 1A, taken along line 1C-1C, showing the container, the outflow line, the return line, the filter, and the defoamer.

FIG. 1D is a front view of the filter of the fluid reservoir of FIG. 1A in accordance with one or more embodiments of the present disclosure.

FIG. 1E is a front view of the filter of the fluid reservoir of FIG. 1A in accordance with one or more embodiments of the present disclosure.

FIG. 1F is a front view of the filter of the fluid reservoir of FIG. 1A in accordance with one or more embodiments of the present disclosure.

FIG. 1G is a front view of the filter of the fluid reservoir of FIG. 1A in accordance with one or more embodiments of the present disclosure.

FIG. 2A is a front view of a fluid reservoir in accordance with one or more embodiments of the present disclosure, showing a container, a number of inflow lines, a vent line, an auxiliary line, an outflow line, and a return line of the fluid reservoir.

FIG. 2B is a detailed cross-sectional bottom view of the fluid reservoir of FIG. 2A, taken along line 2B-2B, showing the container, the inflow lines, the vent line, the auxiliary line, and a filter and a defoamer of the fluid reservoir disposed within an interior space of the container.

FIG. 2C is a detailed cross-sectional top view of the fluid reservoir of FIG. 2A, taken along line 2C-2C, showing the container, the outflow line, the return line, the filter, and the defoamer.

FIG. 3A is a front view of a fluid reservoir in accordance with one or more embodiments of the present disclosure, showing a container, a number of inflow lines, a vent line, a number of auxiliary lines, a number of outflow lines, and a return line of the fluid reservoir.

FIG. 3B is a detailed cross-sectional bottom view of the fluid reservoir of FIG. 3A, taken along line 3B-3B, showing the container, the inflow lines, the vent line, the auxiliary lines, and a filter and a defoamer of the fluid reservoir disposed within an interior space of the container.

FIG. 3C is a detailed cross-sectional top view of the fluid reservoir of FIG. 3A, taken along line 3C-3C, showing the container, the outflow lines, the return line, the filter, and the defoamer.

FIG. 4A is a front view of a fluid reservoir in accordance with one or more embodiments of the present disclosure, showing a container, a number of inflow lines, a vent line, a number of auxiliary lines, a number of outflow lines, and a return line of the fluid reservoir.

FIG. 4B is a detailed cross-sectional bottom view of the fluid reservoir of FIG. 4A, taken along line 4B-4B, showing the container, the inflow lines, the vent line, the auxiliary lines, and a filter and a defoamer of the fluid reservoir disposed within an interior space of the container.

FIG. 4C is a detailed cross-sectional top view of the fluid reservoir of FIG. 4A, taken along line 4C-4C, showing the container, the outflow lines, the return line, the filter, and the defoamer.

FIG. 5A is a front view of a fluid reservoir in accordance with one or more embodiments of the present disclosure, showing a container, a number of inflow lines, a vent line, a number of auxiliary lines, an outflow line, and a return line of the fluid reservoir.

FIG. 5B is a detailed front view of a portion of the fluid reservoir of FIG. 5A, showing the container, the inflow lines, the vent line, the auxiliary lines, the outflow line, the return line, and a filter of the fluid reservoir disposed within an interior space of the container.

FIG. 5C is a detailed cross-sectional bottom view of the fluid reservoir of FIG. 5A, taken along line 5C-5C, showing the container, the inflow lines, the vent line, the auxiliary lines, and the filter.

FIG. 5D is a detailed cross-sectional top view of the fluid reservoir of FIG. 5A, taken along line 5D-5D, showing the container, the outflow line, and the return line.

FIG. 6A is a front view of a fluid reservoir in accordance with one or more embodiments of the present disclosure, showing a container, a number of inflow lines, a vent line, a number of auxiliary lines, an outflow line, and a return line of the fluid reservoir.

FIG. 6B is a detailed front view of a portion of the fluid reservoir of FIG. 6A, showing the container, the inflow lines, the vent line, the auxiliary lines, the outflow line, the return line, and a filter of the fluid reservoir disposed within an interior space of the container.

FIG. 6C is a detailed cross-sectional bottom view of the fluid reservoir of FIG. 6A, taken along line 6C-6C, showing the container, the inflow lines, the vent line, the auxiliary lines, and the filter.

FIG. 6D is a detailed cross-sectional top view of the fluid reservoir of FIG. 6A, taken along line 6D-6D, showing the container, the outflow line, and the return line.

FIG. 7A is a front view of a fluid reservoir in accordance with one or more embodiments of the present disclosure, showing a container, a number of inflow lines, a vent line, a number of auxiliary lines, an outflow line, and a return line of the fluid reservoir.

FIG. 7B is a detailed front view of a portion of the fluid reservoir of FIG. 7A, showing the container, the inflow lines, the vent line, the auxiliary lines, the outflow line, the return line, and a first filter and a second filter of the fluid reservoir disposed within an interior space of the container.

FIG. 7C is a detailed cross-sectional bottom view of the fluid reservoir of FIG. 7A, taken along line 7C-7C, showing the container, the inflow lines, the vent line, the auxiliary lines, the first filter, and the second filter.

FIG. 7D is a detailed cross-sectional top view of the fluid reservoir of FIG. 7A, taken along line 7D-7D, showing the container, the outflow line, and the return line.

### DETAILED DESCRIPTION OF THE DISCLOSURE

Various embodiments of the present disclosure provide improved fluid reservoirs and related methods for use in medical procedures to collect, store, and transfer biological fluids. Such fluid reservoirs and related methods may address one or more of the above-described potential drawbacks of existing technology for collecting, storing, and transferring biological fluids. In certain embodiments, the fluid reservoirs may be collapsible and may include a flexible container and a flexible filter disposed within an interior space of the container. The filter may span the interior space and be attached to the container along an entire periphery of the filter. In this manner, the filter may divide the interior space into two portions, an inflow portion upstream of the filter, and an outflow portion downstream of the filter. Multiple inflow lines may be provided to deliver a biological fluid into the inflow portion of the interior space, and an outflow line may be provided to receive the filtered biological fluid from the outflow portion of the interior space. The fluid reservoirs may be used in an upright (i.e., vertical) orientation with the filter extending in a vertical manner and the inflow lines disposed above the outflow line.

Embodiments of the present disclosure are described herein below with reference to the accompanying drawings, in which some, but not all, embodiments are shown. Indeed, the fluid reservoirs and methods disclosed may be embodied in many different forms and should not be construed as limited to the embodiments set forth herein; rather, these embodiments are provided so that this disclosure is thorough and complete and fully conveys the scope of the fluid reservoirs and methods to those skilled in the art. Like reference numbers refer to like elements throughout. The singular forms “a,” “an,” and “the” can refer to plural instances unless the context clearly dictates otherwise or unless explicitly stated.

As described in detail below, the embodiments of the present disclosure provide improved fluid reservoirs and related methods for use in medical procedures to collect, store, and transfer biological fluids. For example, the fluid reservoirs may be used as a part of an extracorporeal circuit to collect, contain, and/or deliver blood to a patient, although other uses of the fluid reservoirs are envisioned. The fluid reservoirs may be collapsible such that the reservoir adjusts to the volume of fluid contained therein. In some embodiments, the fluid reservoirs may include a flexible container, a flexible filter positioned within and spanning an internal space of the container, a plurality of inflow lines for delivering a biological fluid into an inflow portion of the interior space, and an outflow line for receiving filtered biological fluid from an outflow portion of the interior space. During use, the fluid reservoirs may be oriented in a vertical manner, with the filter extending vertically and the inflow lines positioned above the outflow line.

As compared to existing fluid reservoirs for medical procedures, embodiments of the present disclosure may provide greater throughput in view of the multiple inflow lines provided for the fluid reservoir. In this manner, the fluid reservoirs provided herein may collect and/or transfer biological fluid at a faster rate than existing technology and may eliminate the need for additional reservoirs or reduce the number of reservoirs required in certain applications. Additionally, the fluid reservoirs may include a filter having a larger surface area for contacting incoming biological fluid, which may allow the reservoirs to remove undesired materials from the fluid at a faster rate than existing technology. In some embodiments, the filter of the fluid reservoirs may be configured and oriented in a manner that minimizes the likelihood of clot formation or clogging of the filter during use. Certain embodiments of the fluid reservoirs may include a means for recirculating unfiltered biological fluid, such as a return line for delivering fluid upstream of the filter or receiving fluid upstream of the filter. In some embodiments, the fluid reservoirs may include a means for allowing biological fluid to bypass the filter, such as an inflow line positioned downstream of the filter. In this manner, embodiments of the present application may provide fluid reservoirs and related methods which address one or more of the above-described potential drawbacks of existing technology and are able to be used in a broader range of applications for collecting, storing, and transferring biological fluids, such as blood.

### Collapsible Fluid Reservoirs

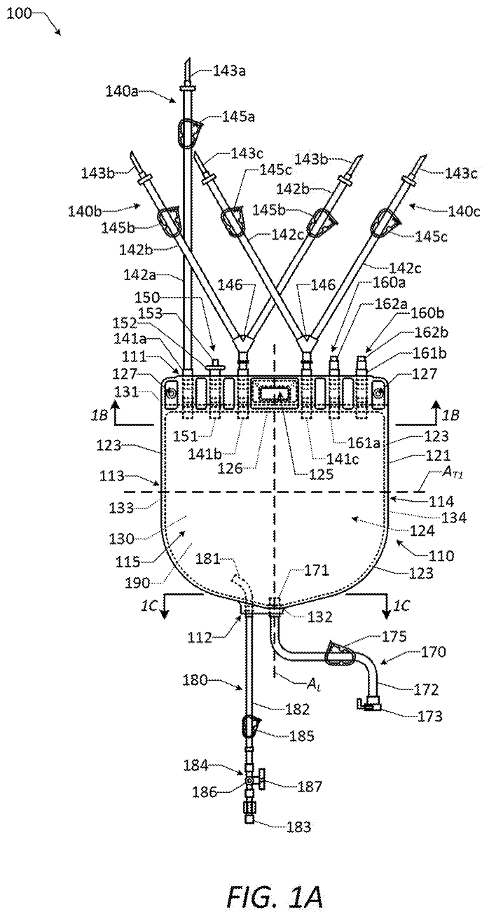

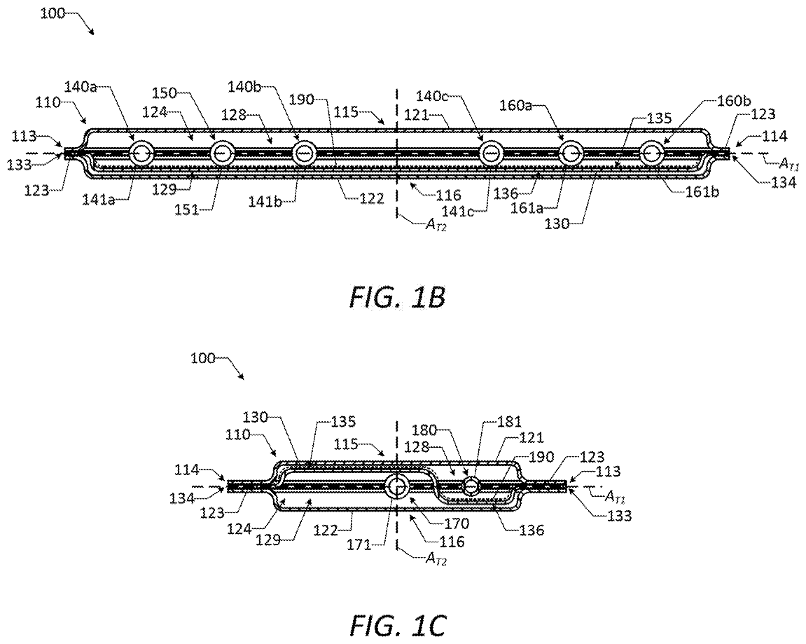

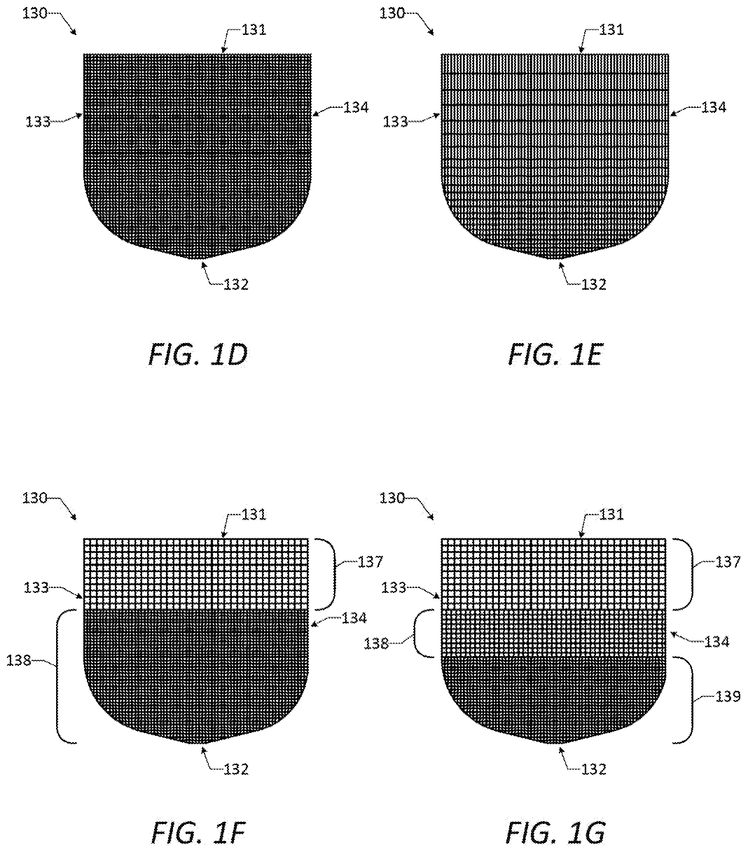

Referring now to the drawings of the present disclosure, FIGS. 1A-1G illustrate a collapsible fluid reservoir 100 (which also may be referred to as a “medical fluid reservoir,” a “fluid reservoir” or simply a “reservoir”) and components thereof according to one or more embodiments of the disclosure. The collapsible fluid reservoir 100 may be configured for use in medical procedures to collect, store, and transfer biological fluids. As described below, the collapsible fluid reservoir 100 may include a flexible container defining an interior space therein, one or more inflow lines for directing a biological fluid into the interior space, and one or more outflow lines for receiving the biological fluid from the interior space. The collapsible fluid reservoir 100 also may include a flexible filter disposed within the interior space and configured for removing materials from the biological fluid. In this manner, the filtered biological material may be provided to the one or more outflow lines for use in a medical procedure. Ultimately, the collapsible fluid reservoir 100 may be used in various procedures for collecting, storing, and transferring biological materials, such as blood, while avoiding certain drawbacks presented by existing fluid reservoirs. According to the illustrated embodiment, the collapsible fluid reservoir 100 may include a flexible container 110, a flexible filter 130, a plurality of inflow lines 140, one or more vent lines 150, one or more auxiliary lines 160, one or more outflow lines 170, and one or more return lines 180.

The flexible container 110 (which also may be referred to as a “bag,” a “shell,” or a “container”) may be configured to receive and contain a biological fluid, such as blood, therein. The container 110 may have an elongated, substantially planar shape, as shown in FIGS. 1A-1C, although the container 110 may be flexed or deformed into a variety of different shapes. As shown, the container 110 may have a first end 111 (which also may be referred to as a “top end”) and a second end 112 (which also may be referred to as a “bottom end”) positioned opposite one another in a direction of a longitudinal axis A_{L} of the container 110. In this manner, the container 110 may have an overall length extending from the first end 111 to the second end 112. The container 110 also may have a first side 113 (which also may be referred to as a “first lateral side”) and a second side 114 (which also may be referred to as a “second lateral side”) positioned opposite one another in a direction of a first transverse axis A_{T1} of the container 110 oriented perpendicular to the longitudinal axis A_{L}. In this manner, the container 110 may have an overall width extending from the first side 113 to the second side 114. As shown, the container 110 also may have a third side 115 (which also may be referred to as a “front side”) and a fourth side 116 (which also may be referred to as a “back side”) positioned opposite one another in a direction of a second transverse axis A_{T2} of the container 110 oriented perpendicular to the longitudinal axis A_{L} and the first transverse axis A_{T1}. In this manner, the container 110 may have an overall thickness extending from the third side 115 to the fourth side 116. The fluid reservoir 100 may be configured for use in a vertical orientation, as shown in FIG. 1A, with the longitudinal axis A_{L} of the container 110 extending in a vertical or substantially vertical manner and the first transverse axis A_{T1} and the second transverse axis A_{T2} of the container 110 extending in a horizontal or substantially horizontal manner. In this manner, the first end 111 may be positioned above the second end 112 during use of the fluid reservoir 100. The flexible container 110 may be provided in different sizes for use in a variety of different applications. In this manner, different sizes of the flexible container 110 may be used depending on a volume of biological fluid to be contained within the interior space 124 thereof. In various embodiments, the volume of the interior space 124 of the container 110 may be between 100 mL and 6000 mL, between 100 mL and 500 mL, between 500 mL and 1000 mL, between 1000 mL and 2000 mL, between 2000 mL and 3000 mL, between 3000 mL and 4000 mL, between 4000 mL and 5000 mL, or between 5000 mL and 6000 mL, although other volumes of the interior space 124 may be used.

As shown, the container 110 may include a pair of flexible sheets attached to one another. In particular, the container 110 may include a first sheet 121 (which also may be referred to as a “first container sheet”) and a second sheet 122 (which also may be referred to as a “second container sheet”) formed of a flexible material and attached to one another. In certain embodiments, the flexible sheets 121, 122 may be formed of a plastic, such as polyvinyl chloride, although other suitable materials may be used. As shown, the sheets 121, 122 may be attached to one another along the outer periphery of the container 110 and may be unattached to one another along an interior region inward of the periphery of the container 110. In certain embodiments, the sheets 121, 122 may be fixedly attached to one another by a seal 123 (which also may be referred to as a “container seal”) formed along the outer periphery of the container, for example, by radio frequency (RF) welding, heat sealing, one or more adhesives, or other means of attachment. As shown, the first sheet 121 and the second sheet 122 may define an interior space 124 therebetween for receiving a biological fluid. The interior space 124 may be spaced apart from the outer periphery of the seal 123, as shown in FIG. 1A. In this manner, the biological fluid may be contained within the interior space 124 during use of the fluid container 100. Each of the sheets 121, 122 may have an elongated, substantially planar shape, although the sheets 121, 122 may be flexed or deformed into a variety of different shapes. In certain embodiments, as shown, each of the sheets 121, 122 may extend along the entire length of the container 110, along the entire width of the container 110, and along the entire periphery of the container 110. In certain embodiments, the first sheet 121 may extend along the third side 115 of the container 110, and the second sheet 122 may extend along the fourth side 116 of the container 100. Each of the sheets 121, 122 may have a number of linear edges and a number of curvilinear edges, as shown in FIG. 1A, although other configurations of the sheets 121, 122 may be used.

The container 110 may include a number of openings defined therethrough and configured to facilitate use of the fluid reservoir 100. For example, the container 110 may include a central opening 125 positioned centrally between the first side 113 and the second side 114 and near the first end 111 of the container 110. The central opening 125 may extend from the third side 115 to the fourth side 116 of the container 110 and be configured to receive a mating structure, such as a hook or a bar, therethrough. In this manner, the fluid reservoir 100 may be suspended and supported by engagement between the mating structure and the central opening 125 during use of the reservoir 100. In certain embodiments, as shown, the fluid reservoir 100 may include a support member 126 extending around and encircling the central opening 125. The support member 126 may be disposed between the first sheet 121 and the second sheet 122 and may be more rigid than the container 110. In this manner, the support member 126 may provide structural support to the fluid reservoir 100 around the central opening 125 and prevent or inhibit deformation of the container 110 thereabout when the reservoir 100 is suspended by the mating structure. The container 110 also may include a pair of lateral openings 127 positioned near the first end 111 of the container 110 and either the first side 113 or the second side 114 of the container 110, respectively. The lateral openings 127 also may extend from the third side 115 to the fourth side 116 of the container 110 and be configured to receive a mating structure, such as a hook or a bar, therethrough during use of the fluid reservoir 100. Additional features and functionality of the container 110 will be appreciated from the corresponding figures and the description of the fluid reservoir 100 provided herein. Certain relationships between the container 110 and other components of the fluid reservoir 100 are described below.

The flexible filter 130 (which also may be referred to as a “filter member,” a “filter assembly,” or a “filter”) may be configured to remove one or more materials or substances from a biological fluid during use of the fluid reservoir 100. For example, the fluid reservoir 100 may be configured for use with blood, and the filter 130 may be configured to remove clots and small clumps of platelets and white blood cells that may form during collection and storage of the blood. Alternatively, the fluid reservoir 100 may be used with other types of biological materials, and the filter 130 may be configured to remove undesired materials from the biological material collected therein. The filter 130 may have an elongated, substantially planar shape, as shown, although the filter 130 may be flexed or deformed into a variety of different shapes. As shown, the filter 130 may have a first end 131 (which also may be referred to as a “top end”) and a second end 132 (which also may be referred to as a “bottom end”) positioned opposite one another in the direction of the longitudinal axis A_{L} of the container 110, a first side 133 (which also may be referred to as a “first lateral side”) and a second side 134 (which also may be referred to as a “second lateral side”) positioned opposite one another in the direction of the first transverse axis A_{T1} of the container 110, and a third side 135 (which also may be referred to as a “front side”) and a fourth side 136 (which also may be referred to as a “back side”) positioned opposite one another in the direction of the second transverse axis A_{T2} of the container 110. As described above, the fluid reservoir 100 may be configured for use in a vertical orientation, as shown in FIG. 1A, with the first end 131 positioned above the second end 132 during use of the reservoir 100.

In certain embodiments, as shown, the filter 130 may be formed as a single sheet of filter material. In other embodiments, the filter 130 may include two or more sheets of filter material attached to one another. In certain embodiments, the filter 130 may be formed of a plastic, although other suitable materials may be used. As shown, the filter 130 may include a plurality of pores defined therein and configured to allow the biological material to pass therethrough while removing the undesired materials. According to various embodiments, the filter 130 may have a pore size between 1 micron and 1000 microns, between 1 micron and 500 microns, between 200 and 250 microns, or about 230 microns. In certain embodiments, the filter 130 may have a pore size that is constant or substantially constant throughout the filter 130. For example, the filter 130 may have a pore size that is constant in the direction of the longitudinal axis A_{L} of the container 110 and in the direction of the first transverse axis A_{T1} of the container 110, as shown in FIG. 1D. In certain embodiments, the filter 130 may have a pore size that varies throughout the filter 130. For example, the filter 130 may have a pore size that decreases in a direction from the first end 131 to the second end 132 of the filter 130 over at least a portion of the length of the filter 130, as shown in FIG. 1E. As another example, the filter 130 may have a pore size that increases in a direction from the first end 131 to the second end 132 of the filter 130 over at least a portion of the length of the filter 130. In certain embodiments, the filter 130 may include two or more regions each having a different, constant or substantially constant pore size throughout the respective region. For example, the filter 130 may include a first region 137 having a first pore size throughout the first region 137, and a second region 138 having a second pore size throughout the second region 138, where the first pore size is different than the second pore size, as shown in FIG. 1F. In certain embodiments, the first pore size may be greater than the second pore size, although the first pore size may be less than the second pore size in other embodiments. In certain embodiments, the first region 137 may extend from the first end 131 of the filter 130 to the second region 138, and the second region 138 may extend from the second end 132 of the filter 130 to the first region 137, although other arrangements of the regions may be used. In certain embodiments, as shown in FIG. 1G, the filter 130 may include a first region 137 having a first pore size throughout the first region 137, a second region 138 having a second pore size throughout the second region 138, and a third region 139 having a third pore size throughout the third region 139, where the first pore size, the second pore size, and the third pore size are different than one another. In certain embodiments, the first pore size may be greater than the second pore size, and the second pore size may be greater than the third pore size. In certain embodiments, the first region 137 may extend from the first end 131 of the filter 130 to the second region 138, the second region 138 may extend from the first region 137 to the third region 139, and the third region 139 may extend from the second end 132 of the filter 130 to the second region 138, although other arrangements of the regions may be used. In certain embodiments, the different regions of the filter 130 may be integrally formed with one another. In other embodiments, the different regions of the filter 130 may be separately formed and attached, either fixedly or removably, to one another.

As shown in FIGS. 1A-1C, the filter 130 may be disposed at least partially within the interior space 124 of the container 110. In certain embodiments, as shown, the filter 130 may be attached to the container 110 along the entire outer periphery of the filter 130. For example, the filter 130 may be fixedly and directly attached to at least one of the first sheet 121 and the second sheet 122 along the entire periphery of the filter 130 by RF welding, heat sealing, one or more adhesives, or other means of attachment. In certain embodiments, as shown, the filter 130 may extend from the first side 113 to the second side 114 of the container 110. In other words, the first side 133 of the filter 130 may be aligned with the first side 113 of the container 110, and the second side 134 of the filter 130 may be aligned with the second side 114 of the container 110. In other embodiments, the sides 133, 134 of the filter 130 may be offset inwardly from the respective sides 113, 114 of the container 110. In certain embodiments, as shown, the first end 131 of the filter 130 may be offset inwardly from the first end 111 of the container 110, and the second end 132 of the filter 130 may be offset inwardly from the second end 112 of the container 110. In other embodiments, the first end 131 of the filter 130 may be aligned with the first end 111 of the container 110, and/or the second end 132 of the filter 130 may be aligned with the second end 112 of the container 110.

As shown, the filter 130 may span the interior space 124 in the direction of the longitudinal axis A_{L} of the container 110 and in the direction of the first transverse axis A_{T1} of the container 110. In this manner, the filter 130 may divide the interior space 124 into two separate portions, with the filter 130 extending therebetween. In particular, a first portion 128 (which also may be referred to as an “inflow portion” or an “upstream portion”) of the interior space 124 may be defined between the third side 135 of the filter 130 and the first sheet 121, and a second portion 129 (which also may be referred to as an “outflow portion” or a “downstream portion”) of the interior space 124 may be defined between the fourth side 136 of the filter 130 and the second sheet 122. In this manner, biological fluid may be delivered into the first portion 128, and the filter 130 may remove undesired materials therefrom as the biological fluid passes through the filter 130 from the first portion 128 into the second portion 129 of the interior space 124. As described above, the first sheet 121, the second sheet 122, and the filter 130 may be flexible, and thus these components may flex or deform, respectively, to accommodate a first volume of the biological fluid in the first portion 128 and a second volume of the filtered biological fluid in the second portion 129. Additional features and functionality of the filter 130 will be appreciated from the corresponding figures and the description of the fluid reservoir 100 provided herein. Certain relationships between the filter 130 and other components of the fluid reservoir 100 are described below.

The inflow lines 140 (which also may be referred to as “fluid inflow lines,” “supply lines,” or “inlet lines”) may be attached to the container 110 and configured to direct biological fluid into the interior space 124 thereof. As shown, the inflow lines 140 may be disposed along the first end 111 of the container 110 and at least partially within the interior space 124. In this manner, respective passages of the inflow lines 140 may be in fluid communication with the interior space 124 for delivering the biological fluid thereto. In various embodiments, the fluid reservoir 100 may include any number of inflow lines 140 attached to the container 110 and configured to direct biological fluid into the interior space 124. According to the illustrated embodiment, the fluid reservoir 100 may include a first inflow line 140a, a second inflow line 140b, and a third inflow line 140c fixedly attached to the container 110. Each of the inflow lines 140 may include an inflow port 141, an inflow tube 142, an inflow fitting 143, and an inflow clamp 145.

As shown in FIG. 1A, the first inflow line 140a may include a first inflow port 141a, a first inflow tube 142a, a first inflow fitting 143a, and a first inflow clamp 145a. As shown, at least a portion of the first inflow line 140a may be disposed within the interior space 124, and at least a portion of the first inflow line 140a may be disposed outside of the interior space 124. In particular, at least a portion of the first inflow port 141a, including a first end thereof, may be disposed within the interior space 124, and at least a portion of the first inflow port 141a, including an opposite second end thereof, and the first inflow tube 142a may be disposed outside of the interior space 124 and extend away from the container 110. As shown in FIG. 1B, the portion of the first inflow port 141a disposed within the interior space 124 may be disposed within the first portion 128 thereof. In other words, the portion of the first inflow port 141a disposed within the interior space 124 may be disposed between the third side 135 of the filter 130 and the first sheet 121 of the container 110. In this manner, the first inflow line 140a may be in fluid communication with the first portion 128 of the interior space 124 and configured to direct biological fluid thereto, upstream of the filter 130. The first inflow port 141a may be fixedly attached to the first sheet 121 and/or the second sheet 122, for example, by RF welding, heat sealing, one or more adhesives, or other means of attachment. A first end of the first inflow tube 142a may be fixedly attached to the second end of the first inflow port 141a, and the first inflow tube 142a may extend away from the container 110. An opposite second end of the first inflow tube 142a may have the inflow fitting 143a fixedly attached thereto. The first inflow fitting 143a may be configured for attaching to other components of an extracorporeal circuit. In certain embodiments, as shown, the first inflow fitting 143a may be formed as a spike fitting, although other types of fittings may be used. The first inflow clamp 145a may be disposed about and positioned around a portion of the first inflow tube 142a near the first inflow fitting 143a. The first inflow clamp 145a may be an adjustable clamp configured to be selectively moved between an open position for allowing fluid flow through the first inflow tube 142a and a closed position for preventing fluid flow through the first inflow tube 142a.

The second inflow line 140b may include a second inflow port 141b, a pair of second inflow tubes 142b, a pair of second inflow fittings 143b, and a pair of second inflow clamps 145b. As shown, at least a portion of the second inflow line 140b may be disposed within the interior space 124, and at least a portion of the second inflow line 140b may be disposed outside of the interior space 124. In particular, at least a portion of the second inflow port 141b, including a first end thereof, may be disposed within the interior space 124, and at least a portion of the second inflow port 141b, including an opposite second end thereof, and the second inflow tubes 142b may be disposed outside of the interior space 124 and extend away from the container 110. As shown in FIG. 1B, the portion of the second inflow port 141b disposed within the interior space 124 may be disposed within the first portion 128 thereof. In other words, the portion of the second inflow port 141b disposed within the interior space 124 may be disposed between the third side 135 of the filter 130 and the first sheet 121 of the container 110. In this manner, the second inflow line 140b may be in fluid communication with the first portion 128 of the interior space 124 and configured to direct biological fluid thereto, upstream of the filter 130. The second inflow port 141b may be fixedly attached to the first sheet 121 and/or the second sheet 122, for example, by RF welding, heat sealing, one or more adhesives, or other means of attachment. The second inflow line 140b also may include a coupler fitting 146 fixedly attached to the second end of the second inflow port 141b. Respective first ends of the second inflow tubes 142b also may be fixedly attached to the coupler fitting 146, and the second inflow tubes 142b may extend away from the container 110. In certain embodiments, the coupler fitting 146 may be formed as a Y-fitting, although other types of fittings may be used. Respective opposite second ends of the second inflow tubes 142b may have the respective second inflow fittings 143b fixedly attached thereto. The second inflow fittings 143b may be configured for attaching to other components of an extracorporeal circuit. In certain embodiments, as shown, the second inflow fittings 143b may be formed as spike fittings, although other types of fittings may be used. The respective second inflow clamps 145b may be disposed about and positioned around respective portions of the second inflow tubes 142b near the respective second inflow fittings 143b. The second inflow clamps 145b may be adjustable clamps configured to be selectively moved between an open position for allowing fluid flow through the respective second inflow tubes 142b and a closed position for preventing fluid flow through the respective second inflow tubes 142b.

In a similar manner, the third inflow line 140c may include a third inflow port 141c, a pair of third inflow tubes 142c, a pair of third inflow fittings 143c, a pair of third inflow clamps 145c, and a coupler fitting 146. As shown, at least a portion of the third inflow line 140c may be disposed within the interior space 124, and at least a portion of the third inflow line 140c may be disposed outside of the interior space 124. In particular, at least a portion of the third inflow port 141c, including a first end thereof, may be disposed within the interior space 124, and at least a portion of the third inflow port 141c, including an opposite second end thereof, and the third inflow tubes 142c may be disposed outside of the interior space 124 and extend away from the container 110. As shown in FIG. 1B, the portion of the third inflow port 141c disposed within the interior space 124 may be disposed within the first portion 128 thereof. In other words, the portion of the third inflow port 141c disposed within the interior space 124 may be disposed between the third side 135 of the filter 130 and the first sheet 121 of the container 110. In this manner, the third inflow line 140c may be in fluid communication with the first portion 128 of the interior space 124 and configured to direct biological fluid thereto, upstream of the filter 130. The third inflow port 141c may be fixedly attached to the first sheet 121 and/or the second sheet 122, for example, by RF welding, heat sealing, one or more adhesives, or other means of attachment. The coupler fitting 146 may be fixedly attached to the second end of the third inflow port 141c. Respective first ends of the third inflow tubes 142c also may be fixedly attached to the coupler fitting 146, and the third inflow tubes 142c may extend away from the container 110. In certain embodiments, the coupler fitting 146 may be formed as a Y-fitting, although other types of fittings may be used. Respective opposite second ends of the third inflow tubes 142c may have the respective third inflow fittings 143c fixedly attached thereto. The third inflow fittings 143c may be configured for attaching to other components of an extracorporeal circuit. In certain embodiments, as shown, the third inflow fittings 143c may be formed as spike fittings, although other types of fittings may be used. The respective third inflow clamps 145c may be disposed about and positioned around respective portions of the third inflow tubes 142c near the respective third inflow fittings 143c. The third inflow clamps 145c may be adjustable clamps configured to be selectively moved between an open position for allowing fluid flow through the respective third inflow tubes 142c and a closed position for preventing fluid flow through the respective third inflow tubes 142c. Additional features and functionality of the inflow lines 140 will be appreciated from the corresponding figures and the description of the fluid reservoir 100 provided herein.

The vent line 150 (which also may be referred to as an “air vent line” or an “air outflow line”) may be attached to the container 110 and configured to allow air or other gases to exit the interior space 124 thereof. As shown, the vent line 150 may be disposed along the first end 111 of the container 110 and at least partially within the interior space 124. In this manner, a passage of the vent line 150 may be in fluid communication with the interior space 124 for receiving air or other gases therefrom. In various embodiments, the fluid reservoir 100 may include any number of vent lines 150 attached to the container 110 and configured to allow air or other gases to exit the interior space 124. According to the illustrated embodiment, the fluid reservoir 100 may include only a single vent line 150.

As shown in FIG. 1A, the vent line 150 may include a vent port 151, a vent filter 152, and a vent fitting 153. As shown, at least a portion of the vent line 150 may be disposed within the interior space 124, and at least a portion of the vent line 150 may be disposed outside of the interior space 124. In particular, at least a portion of the vent port 151, including a first end thereof, may be disposed within the interior space 124, and at least a portion of the vent port 151, including an opposite second end thereof, the vent filter 152, and the vent fitting 153 may be disposed outside of the interior space 124 and extend away from the container 110. As shown in FIG. 1B, the portion of the vent port 151 disposed within the interior space 124 may be disposed within the first portion 128 thereof. In other words, the portion of the vent port 151 disposed within the interior space 124 may be disposed between the third side 135 of the filter 130 and the first sheet 121 of the container 110. In this manner, the vent line 150 may be in fluid communication with the first portion 128 of the interior space 124 and configured to receive air or other gases therefrom, upstream of the filter 130. The vent port 151 may be fixedly attached to the first sheet 121 and/or the second sheet 122, for example, by RF welding, heat sealing, one or more adhesives, or other means of attachment. The vent filter 152 may be fixedly attached to the second end of the vent port 151 and may include an air filter configured to remove materials from the air or other gases exiting the interior space 124 of the container 110. In certain embodiments, the air filter may be a hydrophobic filter configured to allow air and other gases to pass therethrough and to prevent fluid, such as the biological fluid, from passing therethrough. In certain embodiments, the vent filter 152 and/or the air filter thereof may be removably attached to the vent port 151. The vent fitting 153 may be attached to the vent filter 152 and configured for attaching to other components. For example, the vent fitting 153 may be formed as a luer fitting for attaching to a vent container for collecting air or other gases exiting the interior space 124 and/or for attaching to a cap or plug for preventing air or other gases from exiting the interior space 124 through the vent line 150. Additional features and functionality of the vent line 150 will be appreciated from the corresponding figures and the description of the fluid reservoir 100 provided herein.

The auxiliary lines 160 (which also may be referred to as “auxiliary fluid inflow lines,” “auxiliary supply lines,” or “auxiliary inlet lines”) may be attached to the container 110 and configured to direct additional fluids into the interior space 124 thereof. As shown, the auxiliary lines 160 may be disposed along the first end 111 of the container 110 and at least partially within the interior space 124. In this manner, respective passages of the auxiliary lines 160 may be in fluid communication with the interior space 124 for delivering the additional fluids thereto. In various embodiments, the fluid reservoir 100 may include any number of auxiliary lines 160 attached to the container 110 and configured to direct additional fluids into the interior space 124. According to the illustrated embodiment, the fluid reservoir 100 may include a first auxiliary line 160a and a second auxiliary line 160b fixedly attached to the container 110. Each of the auxiliary lines 160 may include an auxiliary port 161 and an auxiliary fitting 162.

As shown in FIG. 1A, the first auxiliary line 160a may include a first auxiliary port 161a and a first auxiliary fitting 162a. As shown, at least a portion of the first auxiliary line 160a may be disposed within the interior space 124, and at least a portion of the first auxiliary line 160a may be disposed outside of the interior space 124. In particular, at least a portion of the first auxiliary port 161a, including a first end thereof, may be disposed within the interior space 124, and at least a portion of the first auxiliary port 161a, including an opposite second end thereof, and the first auxiliary fitting 162a may be disposed outside of the interior space 124 and extend away from the container 110. As shown in FIG. 1B, the portion of the first auxiliary port 161a disposed within the interior space 124 may be disposed within the first portion 128 thereof. In other words, the portion of the first auxiliary port 161a disposed within the interior space 124 may be disposed between the third side 135 of the filter 130 and the first sheet 121 of the container 110. In this manner, the first auxiliary line 160a may be in fluid communication with the first portion 128 of the interior space 124 and configured to deliver additional fluids thereto, upstream of the filter 130. The first auxiliary port 161a may be fixedly attached to the first sheet 121 and/or the second sheet 122, for example, by RF welding, heat sealing, one or more adhesives, or other means of attachment. The first auxiliary fitting 162a may be fixedly attached to the second end of the first auxiliary port 161a and configured for attaching to other components. For example, the first auxiliary fitting 162a may be formed as a luer fitting for attaching to a fluid delivery mechanism for delivering additional fluids into the interior space 124 and/or for attaching to a cap or plug for closing the passage of the first auxiliary line 160a.

In a similar manner, the second auxiliary line 160b may include a second auxiliary port 161b and a second auxiliary fitting 162b. As shown, at least a portion of the second auxiliary line 160b may be disposed within the interior space 124, and at least a portion of the second auxiliary line 160b may be disposed outside of the interior space 124. In particular, at least a portion of the second auxiliary port 161b, including a first end thereof, may be disposed within the interior space 124, and at least a portion of the second auxiliary port 161b, including an opposite second end thereof, and the second auxiliary fitting 162b may be disposed outside of the interior space 124 and extend away from the container 110. As shown in FIG. 1B, the portion of the second auxiliary port 161b disposed within the interior space 124 may be disposed within the first portion 128 thereof. In other words, the portion of the second auxiliary port 161b disposed within the interior space 124 may be disposed between the third side 135 of the filter 130 and the first sheet 121 of the container 110. In this manner, the second auxiliary line 160b may be in fluid communication with the first portion 128 of the interior space 124 and configured to deliver additional fluids thereto, upstream of the filter 130. The second auxiliary port 161b may be fixedly attached to the first sheet 121 and/or the second sheet 122, for example, by RF welding, heat sealing, one or more adhesives, or other means of attachment. The second auxiliary fitting 162b may be fixedly attached to the second end of the second auxiliary port 161b and configured for attaching to other components. For example, the second auxiliary fitting 162b may be formed as a luer fitting for attaching to a fluid delivery mechanism for delivering additional fluids into the interior space 124 and/or for attaching to a cap or plug for closing the passage of the second auxiliary line 160b. Additional features and functionality of the auxiliary lines 160 will be appreciated from the corresponding figures and the description of the fluid reservoir 100 provided herein.

The outflow line 170 (which also may be referred to as a “fluid outflow line,” an “exit line,” or an “outlet line”) may be attached to the container 110 and configured to allow biological fluid to exit the interior space 124 thereof. As shown, the outflow line 170 may be disposed along the second end 112 of the container 110 and at least partially within the interior space 124. In this manner, a passage of the outflow line 170 may be in fluid communication with the interior space 124 for receiving biological fluid therefrom. In various embodiments, the fluid reservoir 100 may include any number of outflow lines 170 attached to the container 110 and configured to allow biological fluids to exit the interior space 124. According to the illustrated embodiment, the fluid reservoir 100 may include only a single outflow line 170.

As shown in FIG. 1A, the outflow line 170 may include an outflow port 171, an outflow tube 172, an outflow fitting 173, and an outflow clamp 175. As shown, at least a portion of the outflow line 170 may be disposed within the interior space 124, and at least a portion of the outflow line 170 may be disposed outside of the interior space 124. In particular, at least a portion of the outflow port 171, including a first end thereof, may be disposed within the interior space 124, and at least a portion of the outflow port 171, including an opposite second end thereof, and the outflow tube 172 may be disposed outside of the interior space 124 and extend away from the container 110. In certain embodiments, as shown, the first end of the outflow port 171 may be positioned at or along the bottommost edge of the interior space 124, and the bottommost edge of the interior space 124 may be curved or tapered in a downward manner toward the first end of the outflow port 171. In this manner, the biological fluid within the interior space 124 may naturally move toward the outflow port 171 and be drained therefrom. As shown in FIG. 1C, the portion of the outflow port 171 disposed within the interior space 124 may be disposed within the second portion 129 thereof. In other words, the portion of the outflow port 171 disposed within the interior space 124 may be disposed between the fourth side 136 of the filter 130 and the second sheet 122 of the container 110. In this manner, the outflow line 170 may be in fluid communication with the second portion 129 of the interior space 124 and configured to receive biological fluid therefrom, downstream of the filter 130. The outflow port 171 may be fixedly attached to the first sheet 121 and/or the second sheet 122, for example, by RF welding, heat sealing, one or more adhesives, or other means of attachment. A first end of the outflow tube 172 may be fixedly attached to the second end of the outflow port 171, and the outflow tube 172 may extend away from the container 110. An opposite second end of the outflow tube 172 may have the outflow fitting 173 fixedly attached thereto. The outflow fitting 173 may be configured for attaching to other components of an extracorporeal circuit. In certain embodiments, as shown, the outflow fitting 173 may be formed as a lock fitting for releasably locking onto another component, although other types of fittings may be used. The outflow clamp 175 may be disposed about and positioned around a portion of the outflow tube 172 near the outflow fitting 173. The outflow clamp 175 may be an adjustable clamp configured to be selectively moved between an open position for allowing fluid flow through the outflow tube 172 and a closed position for preventing fluid flow through the outflow tube 172. Additional features and functionality of the outflow line 170 will be appreciated from the corresponding figures and the description of the fluid reservoir 100 provided herein.

The return line 180 (which also may be referred to as a “fluid return line” or a “recirculation line”) may be attached to the container 110 and configured to direct biological fluid into the interior space 124 thereof or to allow biological fluid to exit the interior space 124 thereof. As shown, the return line 180 may be disposed along the second end 112 of the container 110 and at least partially within the interior space 124. In this manner, a passage of the return line 180 may be in fluid communication with the interior space 124 for delivering biological fluid thereto or receiving biological fluid therefrom. In various embodiments, the fluid reservoir 100 may include any number of return lines 180 attached to the container 110 and configured to allow biological fluids to pass into the interior space 124 or to exit the interior space 124. According to the illustrated embodiment, the fluid reservoir 100 may include only a single return line 180.

As shown in FIG. 1A, the return line 180 may include a return port 181, a return tube 182, a return fitting 183, a return valve 184, and a return clamp 185. As shown, at least a portion of the return line 180 may be disposed within the interior space 124, and at least a portion of the return line 180 may be disposed outside of the interior space 124. In particular, at least a portion of the return port 181, including a first end thereof, may be disposed within the interior space 124, and at least a portion of the return port 181, including an opposite second end thereof, and the return tube 182 may be disposed outside of the interior space 124 and extend away from the container 110. As shown in FIG. 1B, the portion of the return port 181 disposed within the interior space 124 may be disposed within the first portion 128 thereof. In other words, the portion of the return port 181 disposed within the interior space 124 may be disposed between the third side 135 of the filter 130 and the first sheet 121 of the container 110. In this manner, the return line 180 may be in fluid communication with the first portion 128 of the interior space 124 and configured to deliver biological fluid thereto or receive biological fluid therefrom, upstream of the filter 130. The return port 181 may be fixedly attached to the first sheet 121 and/or the second sheet 122, for example, by RF welding, heat sealing, one or more adhesives, or other means of attachment. In certain embodiments, as shown, the return port 181 may be positioned along the second end 112 of the container 110, although other positions of the return port 181 may be used. In certain embodiments, the return port 181 may be spaced apart from the second end 112 of the container 110. For example, the return port 181 may be positioned along one of the first side 113, the second side 114, the third side 115, or the fourth side 116 of the container 110 and spaced apart from the second end 112 thereof. In certain embodiments, the return port 181 may have a curved or angled shape that extends away from the outflow port 171. For example, the return port 181 may curve away from the outflow port 171 toward the first side 113 or the second side 114 of the container 110. A first end of the return tube 182 may be fixedly attached to the second end of the return port 181, and the return tube 182 may extend away from the container 110. An opposite second end of the return tube 182 may have the return fitting 183 fixedly attached thereto. The return fitting 183 may be configured for attaching to other components of an extracorporeal circuit. For example, the return fitting 183 may be attached to one or more additional lines and/or fittings such that the return line 180 is in fluid communication with one or more of the inflow lines 140. In this manner, unfiltered biological fluid from the first portion 128 of the interior space 124 may be recirculated through the return line 180 and/or one or more of the inflow lines 140. In certain embodiments, as shown, the return fitting 183 may be formed as a luer fitting for releasably attaching to another component, although other types of fittings may be used. The return valve 184 may be positioned between the return tube 182 and the return fitting 183 and configured to control fluid flow through the return line 180. In certain embodiments, as shown, the return valve 184 may be a three-way valve movable between a first position allowing fluid flow from or to the return fitting 183, a second position allowing fluid flow from or to an additional outlet 186 thereof, a third position allowing fluid flow from or to both the return fitting 183 and the additional outlet 186, and a fourth position preventing fluid flow from or to both the return fitting 183 and the additional outlet 186. In certain embodiments, the return valve 184 also may include a knob 187 for moving the valve 184 between the different positions. The return clamp 185 may be disposed about and positioned around a portion of the return tube 182 near the return valve 184. The return clamp 185 may be an adjustable clamp configured to be selectively moved between an open position for allowing fluid flow through the return tube 182 and a closed position for preventing fluid flow through the return tube 182. Additional features and functionality of the return line 180 will be appreciated from the corresponding figures and the description of the fluid reservoir 100 provided herein.

In certain embodiments, the fluid reservoir 100 also may include a defoamer 190 (which also may be referred to as a “defoamer member” or an “anti-foaming member”), illustrated via dashed lines in the figures. The defoamer 190 may be configured to break up or reduce air or other gas bubbles that may form in the biological fluid contained within the container 110. The defoamer 190 may be formed of various materials and may have a cellular or porous configuration. In certain embodiments, the defoamer 190 may include one or more defoaming or anti-foaming agents to facilitate the reduction of bubbles in the biological fluid. In certain embodiments, the defoamer 190 may be configured for use with blood or other types of biological materials. In certain embodiments, the defoamer 190 may be formed as one or more flexible sheets of material.

As shown in FIGS. 1A-1C, the defoamer 190 may be disposed at least partially within the interior space 124 of the container 110. In certain embodiments, the defoamer 190 may be positioned adjacent to the filter 130 within the interior space 124. In certain embodiments, the defoamer 190 may have the same size and shape as the filter 130. In certain embodiments, similar to the filter 130, the defoamer 190 may be attached to the container 110 along the entire outer periphery of the defoamer 190. For example, the defoamer 190 may be fixedly and directly attached to at least one of the first sheet 121 and the second sheet 122 along the entire periphery of the defoamer 190 by RF welding, heat sealing, one or more adhesives, or other means of attachment. In certain embodiments, as shown, the defoamer 190 may extend from the first side 113 to the second side 114 of the container 110. In other embodiments, the defoamer 190 may be directly attached to the container 110 along only a portion of the periphery of the defoamer 190, or the defoamer 190 may not be directly attached to the container 110 at all. In certain embodiments, the defoamer 190 may be directly attached to the filter 130 along one or more portions thereof. In still other embodiments, the defoamer 190 may not be directly attached to the container 110 or the filter 130, but may be captured within the interior space 124 between the filter 130 and one of the first sheet 121 and the second sheet 122. In certain embodiments, as shown, the defoamer 190 may span the interior space 124 in the direction of the longitudinal axis A_{L} of the container 110 and in the direction of the first transverse axis A_{T1} of the container 110. In certain embodiments, the defoamer 190 may be disposed within the first portion 128 of the interior space 124, between the filter 130 and the first sheet 121 of the container 110. In this manner, the defoamer 190 may remove bubbles from biological fluid before the biological fluid passes through the filter 130 from the first portion 128 to the second portion 129 of the interior space 124. As described above, the defoamer 190 may be formed of a flexible material, and thus the defoamer 190 may flex or deform to accommodate changes in the first volume of biological fluid in the first portion 128 and the second volume of the filtered biological fluid in the second portion 129. Additional features and functionality of the defoamer 190 will be appreciated from the corresponding figures and the description of the fluid reservoir 100 provided herein. In certain embodiments, the defoamer 190 may be omitted form the fluid reservoir 100.

In certain embodiments, the fluid reservoir 100 may be assembled by sealing or welding components of the reservoir 100 to one another. In certain embodiments, the assembly process may begin by positioning the first sheet 121, the second sheet 122, the support member 126, the filter 130, the inflow ports 141a, 141b, 141c, the vent port 151, the auxiliary ports 161a, 161b, the outflow port 171, and the return port 181 as shown in FIGS. 1A-1C. The seal 123 then may be formed, for example, by heat sealing or RF welding, along the respective outer peripheries of the first sheet 121, the second sheet 122, and the filter 130. In this manner, the first sheet 121, the second sheet 122, the filter 130, the inflow ports 141a, 141b, 141c, the vent port 151, the auxiliary ports 161a, 161b, the outflow port 171, and the return port 181 may be fixedly attached to one another by the seal 123. In certain embodiments, the first sheet 121 and the second sheet 122 may be directly attached to one another along one or more first portions of the seal 123, and the first sheet 121 and the second sheet 122 may be indirectly attached to one another along one or more second portions of the seal 123. For example, according to the illustrated embodiment, the first sheet 121 and the second sheet 122 may be directly attached to one another along respective first portions of the seal 123 positioned along the first end 111 of the container 110. However, the first sheet 121 and the second sheet 122 may be indirectly attached to one another via other components of the fluid reservoir 100 along respective second portions of the seal 123 where the other components are positioned between the first sheet 121 and the second sheet 122. As shown, the first sheet 121 and the second sheet 122 may be indirectly attached to one another via the filter 130 along respective second portions of the seal 123 positioned along the periphery of the filter 130. Additionally, the first sheet 121 and the second sheet 122 may be indirectly attached to one another via the support member 126, the filter 130, the inflow ports 141a, 141b, 141c, the vent port 151, the auxiliary ports 161a, 161b, the outflow port 171, and/or the return port 181 along respective second portions of the seal 123 where respective portions of such components are positioned between the first sheet 121 and the second sheet 122, as shown. In certain embodiments, the remaining components of the inflow lines 140a, 140b, 140c, the vent line 150, the auxiliary lines 160a, 160b, the outflow line 170, and the return line 180 may be attached prior to forming the seal 123. In other embodiments, such components may be attached after forming the seal 123.

During use, the fluid reservoir 100 may be oriented in a vertical manner, as shown in FIG. 1A, with the reservoir 100 suspended by a mating structure engaging the central opening 125 and/or the lateral openings 127 of the container 110. Biological fluid may be delivered into the interior space 124 of the container 110 via one or more of the inflow lines 140. In particular, unfiltered biological fluid may be delivered through one or more of the inflow lines 140 and into the first portion 128 of the interior space 124. The biological fluid may pass from the first portion 128, through the filter 130, and into the second portion 129 of the interior space 124, as the filter 130 removes undesired materials or substances from the biological fluid. The filtered biological fluid may pass from the second portion 129 of the interior space 124 and through the outflow line 170 for use in a medical procedure. Meanwhile, air or other gases may exit the interior space 124 through the vent line 150. In particular, air or other gases may pass from the first portion 128 of the interior space 124 and through the vent line 150, while the biological fluid is prevented from passing through the vent line 150 by the vent filter 152 thereof. In certain embodiments, the defoamer 190 may break up or reduce bubbles in the biological fluid prior to passing through the filter 130. Additional fluids may be delivered into the interior space 124 via one or more of the auxiliary lines 160. In particular, additional fluids may be delivered through one or more of the auxiliary lines 160 and into the first portion 128 of the interior space 124. Further, unfiltered biological fluid may pass from the first portion 128 of the interior space 124 and into the return line 180 for recirculation to one or more of the inflow lines 140. Alternatively, recirculated biological fluid may pass through the return line 180 into the first portion 128 of the interior space 124 to be filtered. Other uses and functional aspects of the fluid reservoir 100 will be appreciated from the corresponding figures and the description above.

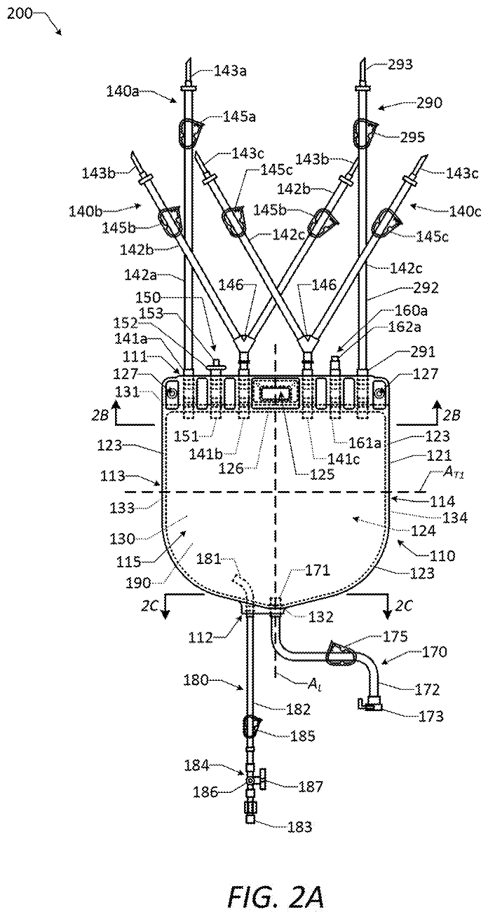

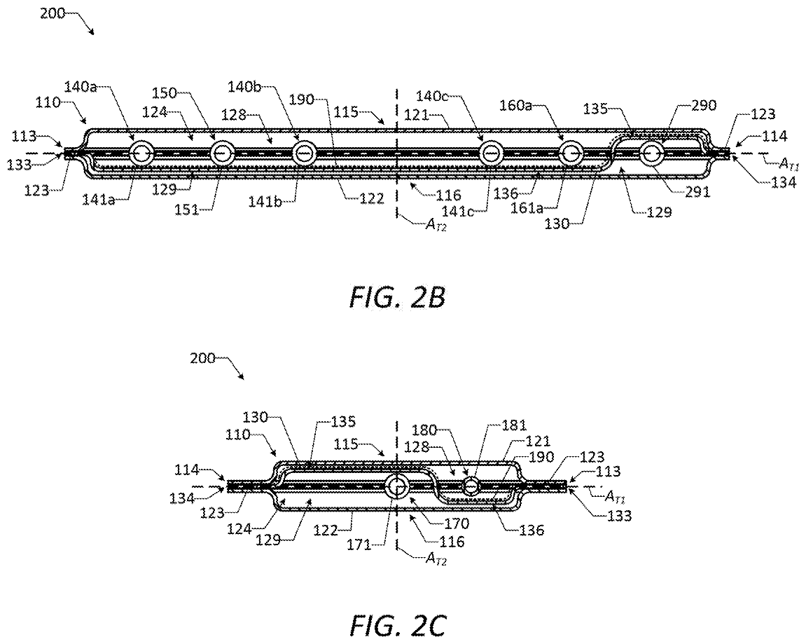

FIGS. 2A-2C illustrate a collapsible fluid reservoir 200 (which also may be referred to as a “medical fluid reservoir,” a “fluid reservoir” or simply a “reservoir”) and components thereof according to one or more embodiments of the disclosure. The collapsible fluid reservoir 200 may be configured for use in medical procedures to collect, store, and transfer biological fluids, such as blood. As shown, the collapsible fluid reservoir 200 generally may be configured in a manner similar to the collapsible fluid reservoir 100 described above, although certain differences are noted below. Like reference numbers indicate similar or identical components or features.

As shown, the collapsible fluid reservoir 200 may include the first inflow line 140a, the second inflow line 140b, and the third inflow line 140c configured in the manner described above. The fluid reservoir 200 also may include a fourth inflow line 290 (which also may be referred to as a “fluid inflow line,” a “supply line,” or an “inlet line”) attached to the container 110 and configured to direct biological fluid into the interior space 124 thereof. As shown, the fourth inflow line 290 may be disposed along the first end 111 of the container 110 and at least partially within the interior space 124. In this manner, a passage of the fourth inflow line 290 may be in fluid communication with the interior space 124 for delivering the biological fluid thereto. In various embodiments, the fluid reservoir 200 may include any number of inflow lines attached to the container 110 and configured to direct biological fluid into the interior space 124.

As shown in FIG. 2A, the fourth inflow line 290 may include a fourth inflow port 291, a fourth inflow tube 292, a fourth inflow fitting 293, and a fourth inflow clamp 295. As shown, at least a portion of the fourth inflow line 290 may be disposed within the interior space 124, and at least a portion of the fourth inflow line 290 may be disposed outside of the interior space 124. In particular, at least a portion of the fourth inflow port 291, including a first end thereof, may be disposed within the interior space 124, and at least a portion of the fourth inflow port 291, including an opposite second end thereof, and the fourth inflow tube 292 may be disposed outside of the interior space 124 and extend away from the container 110. As shown in FIG. 2B, the portion of the fourth inflow port 291 disposed within the interior space 124 may be disposed within the second portion 129 thereof. In other words, the portion of the fourth inflow port 291 disposed within the interior space 124 may be disposed between the fourth side 136 of the filter 130 and the second sheet 122 of the container 110. In this manner, the fourth inflow line 290 may be in fluid communication with the second portion 129 of the interior space 124 and configured to direct biological fluid thereto, downstream of the filter 130. The fourth inflow port 291 may be fixedly attached to the first sheet 121 and/or the second sheet 122, for example, by RF welding, heat sealing, one or more adhesives, or other means of attachment. A first end of the fourth inflow tube 292 may be fixedly attached to the second end of the fourth inflow port 291, and the fourth inflow tube 292 may extend away from the container 110. An opposite second end of the fourth inflow tube 292 may have the inflow fitting 293 fixedly attached thereto. The fourth inflow fitting 293 may be configured for attaching to other components of an extracorporeal circuit. In certain embodiments, as shown, the fourth inflow fitting 293 may be formed as a spike fitting, although other types of fittings may be used. The fourth inflow clamp 295 may be disposed about and positioned around a portion of the fourth inflow tube 292 near the fourth inflow fitting 293. The fourth inflow clamp 295 may be an adjustable clamp configured to be selectively moved between an open position for allowing fluid flow through the fourth inflow tube 292 and a closed position for preventing fluid flow through the fourth inflow tube 292.

In certain embodiments, the fluid reservoir 200 may be assembled by sealing or welding components of the reservoir 200 to one another. In certain embodiments, the assembly process may begin by positioning the first sheet 121, the second sheet 122, the support member 126, the filter 130, the inflow ports 141a, 141b, 141c, 291, the vent port 151, the auxiliary port 161a, the outflow port 171, and the return port 181 as shown in FIGS. 2A-2C. The seal 123 then may be formed, for example, by heat sealing or RF welding, along the respective outer peripheries of the first sheet 121, the second sheet 122, and the filter 130. In this manner, the first sheet 121, the second sheet 122, the filter 130, the inflow ports 141a, 141b, 141c, 291, the vent port 151, the auxiliary port 161a, the outflow port 171, and the return port 181 may be fixedly attached to one another by the seal 123. In certain embodiments, the first sheet 121 and the second sheet 122 may be directly attached to one another along one or more first portions of the seal 123, and the first sheet 121 and the second sheet 122 may be indirectly attached to one another along one or more second portions of the seal 123. For example, according to the illustrated embodiment, the first sheet 121 and the second sheet 122 may be directly attached to one another along respective first portions of the seal 123 positioned along the first end 111 of the container 110. However, the first sheet 121 and the second sheet 122 may be indirectly attached to one another via other components of the fluid reservoir 200 along respective second portions of the seal 123 where the other components are positioned between the first sheet 121 and the second sheet 122. As shown, the first sheet 121 and the second sheet 122 may be indirectly attached to one another via the filter 130 along respective second portions of the seal 123 positioned along the periphery of the filter 130. Additionally, the first sheet 121 and the second sheet 122 may be indirectly attached to one another via the support member 126, the filter 130, the inflow ports 141a, 141b, 141c, 291, the vent port 151, the auxiliary port 161a, the outflow port 171, and/or the return port 181 along respective second portions of the seal 123 where respective portions of such components are positioned between the first sheet 121 and the second sheet 122, as shown. In certain embodiments, the remaining components of the inflow lines 140a, 140b, 140c, 290, the vent line 150, the auxiliary line 160a, the outflow line 170, and the return line 180 may be attached prior to forming the seal 123. In other embodiments, such components may be attached after forming the seal 123.

During use, the fluid reservoir 200 may be oriented in a vertical manner, as shown in FIG. 2A, with the reservoir 200 suspended by a mating structure engaging the central opening 125 and/or the lateral openings 127 of the container 110. Biological fluid may be delivered into the interior space 124 of the container 110 via one or more of the inflow lines 140. In particular, unfiltered biological fluid may be delivered through one or more of the inflow lines 140 and into the first portion 128 of the interior space 124. The biological fluid may pass from the first portion 128, through the filter 130, and into the second portion 129 of the interior space 124, as the filter 130 removes undesired materials or substances from the biological fluid. The filtered biological fluid may pass from the second portion 129 of the interior space 124 and through the outflow line 170 for use in a medical procedure. Additionally or alternatively, biological fluid may be delivered into the interior space 124 of the container 110 via the fourth inflow line 290. In particular, already filtered biological fluid or biological fluid that does not need to be filtered may be delivered through the fourth inflow line 290 and into the second portion 129 of the interior space 124, thereby bypassing the filter 130. The biological fluid subsequently may pass from the second portion 129 of the interior space 124 and through the outflow line 170 for use in a medical procedure. Other uses and functional aspects of the fluid reservoir 200 will be appreciated from the corresponding figures and the description above.

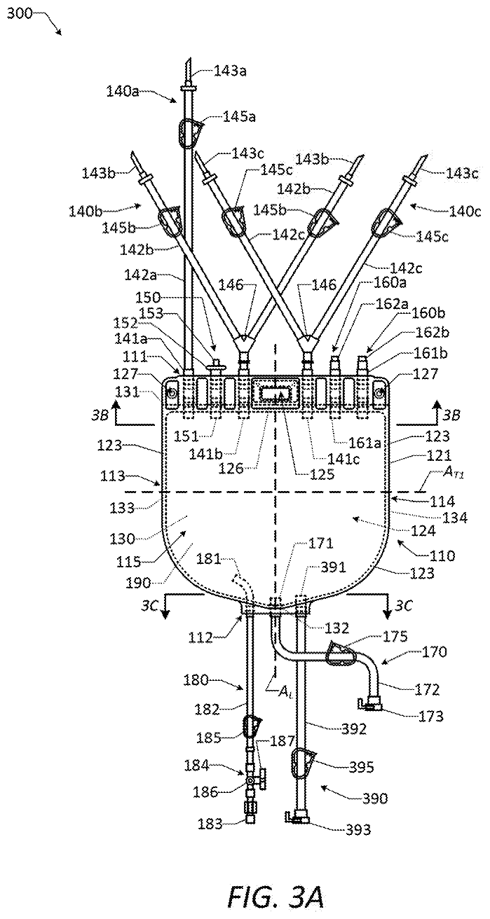

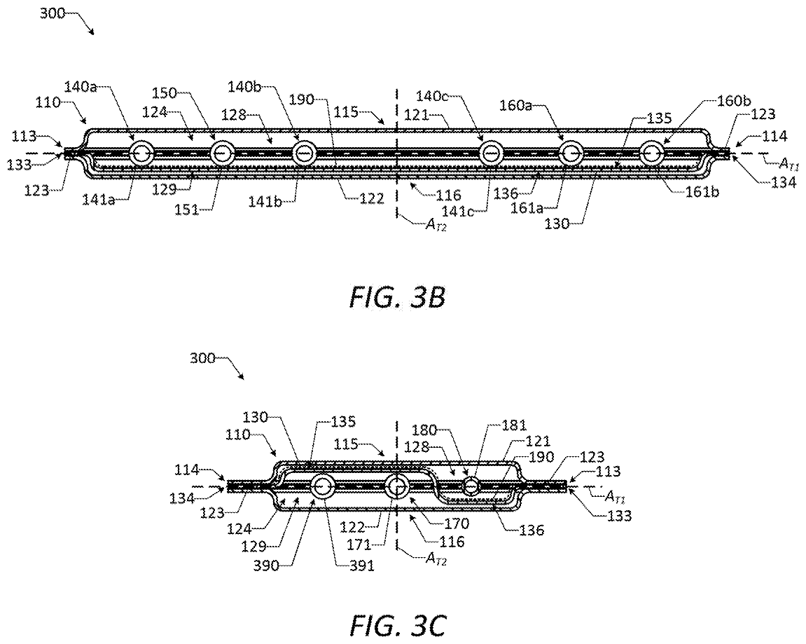

FIGS. 3A-3C illustrate a collapsible fluid reservoir 300 (which also may be referred to as a “medical fluid reservoir,” a “fluid reservoir” or simply a “reservoir”) and components thereof according to one or more embodiments of the disclosure. The collapsible fluid reservoir 300 may be configured for use in medical procedures to collect, store, and transfer biological fluids, such as blood. As shown, the collapsible fluid reservoir 300 generally may be configured in a manner similar to the collapsible fluid reservoir 100 described above, although certain differences are noted below. Like reference numbers indicate similar or identical components or features.

As shown, the collapsible fluid reservoir 300 may include the outflow line 170 configured in the manner described above. The fluid reservoir 300 also may include a second outflow line 390 (which also may be referred to as a “fluid outflow line,” an “exit line,” or an “outlet line”) attached to the container 110 and configured to allow biological fluid to exit the interior space 124 thereof. As shown, the second outflow line 390 may be disposed along the second end 112 of the container 110 and at least partially within the interior space 124. In this manner, a passage of the second outflow line 390 may be in fluid communication with the interior space 124 for receiving biological fluid therefrom. In various embodiments, the fluid reservoir 300 may include any number of outflow lines attached to the container 110 and configured to allow biological fluids to exit the interior space 124.

As shown in FIG. 3A, the second outflow line 390 may include a second outflow port 391, a second outflow tube 392, a second outflow fitting 393, and a second outflow clamp 395. As shown, at least a portion of the second outflow line 390 may be disposed within the interior space 124, and at least a portion of the second outflow line 390 may be disposed outside of the interior space 124. In particular, at least a portion of the second outflow port 391, including a first end thereof, may be disposed within the interior space 124, and at least a portion of the second outflow port 391, including an opposite second end thereof, and the second outflow tube 392 may be disposed outside of the interior space 124 and extend away from the container 110. As shown in FIG. 3C, the portion of the second outflow port 391 disposed within the interior space 124 may be disposed within the second portion 129 thereof. In other words, the portion of the second outflow port 391 disposed within the interior space 124 may be disposed between the fourth side 136 of the filter 130 and the second sheet 122 of the container 110. In this manner, the second outflow line 390 may be in fluid communication with the second portion 129 of the interior space 124 and configured to receive biological fluid therefrom, downstream of the filter 130. The second outflow port 391 may be fixedly attached to the first sheet 121 and/or the second sheet 122, for example, by RF welding, heat sealing, one or more adhesives, or other means of attachment. A first end of the second outflow tube 392 may be fixedly attached to the second end of the second outflow port 391, and the second outflow tube 392 may extend away from the container 110. An opposite second end of the second outflow tube 392 may have the second outflow fitting 393 fixedly attached thereto. The second outflow fitting 393 may be configured for attaching to other components of an extracorporeal circuit. In certain embodiments, as shown, the second outflow fitting 393 may be formed as a lock fitting for releasably locking onto another component, although other types of fittings may be used. The second outflow clamp 395 may be disposed about and positioned around a portion of the second outflow tube 392 near the second outflow fitting 393. The second outflow clamp 395 may be an adjustable clamp configured to be selectively moved between an open position for allowing fluid flow through the second outflow tube 392 and a closed position for preventing fluid flow through the second outflow tube 392.

In certain embodiments, the fluid reservoir 300 may be assembled by sealing or welding components of the reservoir 300 to one another. In certain embodiments, the assembly process may begin by positioning the first sheet 121, the second sheet 122, the support member 126, the filter 130, the inflow ports 141a, 141b, 141c, the vent port 151, the auxiliary ports 161a, 161b, the outflow ports 171, 391, and the return port 181 as shown in FIGS. 3A-3C. The seal 123 then may be formed, for example, by heat sealing or RF welding, along the respective outer peripheries of the first sheet 121, the second sheet 122, and the filter 130. In this manner, the first sheet 121, the second sheet 122, the filter 130, the inflow ports 141a, 141b, 141c, the vent port 151, the auxiliary ports 161a, 162a, the outflow ports 171, 391, and the return port 181 may be fixedly attached to one another by the seal 123. In certain embodiments, the first sheet 121 and the second sheet 122 may be directly attached to one another along one or more first portions of the seal 123, and the first sheet 121 and the second sheet 122 may be indirectly attached to one another along one or more second portions of the seal 123. For example, according to the illustrated embodiment, the first sheet 121 and the second sheet 122 may be directly attached to one another along respective first portions of the seal 123 positioned along the first end 111 of the container 110. However, the first sheet 121 and the second sheet 122 may be indirectly attached to one another via other components of the fluid reservoir 300 along respective second portions of the seal 123 where the other components are positioned between the first sheet 121 and the second sheet 122. As shown, the first sheet 121 and the second sheet 122 may be indirectly attached to one another via the filter 130 along respective second portions of the seal 123 positioned along the periphery of the filter 130. Additionally, the first sheet 121 and the second sheet 122 may be indirectly attached to one another via the support member 126, the filter 130, the inflow ports 141a, 141b, 141c, the vent port 151, the auxiliary ports 161a, 161b, the outflow ports 171, 391, and/or the return port 181 along respective second portions of the seal 123 where respective portions of such components are positioned between the first sheet 121 and the second sheet 122, as shown. In certain embodiments, the remaining components of the inflow lines 140a, 140b, 140c, the vent line 150, the auxiliary lines 160a, 160b, the outflow lines 170, 390, and the return line 180 may be attached prior to forming the seal 123. In other embodiments, such components may be attached after forming the seal 123.

During use, the fluid reservoir 300 may be oriented in a vertical manner, as shown in FIG. 3A, with the reservoir 300 suspended by a mating structure engaging the central opening 125 and/or the lateral openings 127 of the container 110. Biological fluid may be delivered into the interior space 124 of the container 110 via one or more of the inflow lines 140. In particular, unfiltered biological fluid may be delivered through one or more of the inflow lines 140 and into the first portion 128 of the interior space 124. The biological fluid may pass from the first portion 128, through the filter 130, and into the second portion 129 of the interior space 124, as the filter 130 removes undesired materials or substances from the biological fluid. The filtered biological fluid may pass from the second portion 129 of the interior space 124 and through the outflow line 170 for use in a medical procedure. Additionally or alternatively, the filtered biological fluid may pass from the second portion 129 of the interior space 124 and through the second outflow line 390 for use in a medical procedure. The outflow line 170 and the second outflow line 390 may be used simultaneously or at different times to draw desired amounts of the biological fluid from the interior space 124 of the container 110, as may vary during a medical procedure and between different medical procedures. Other uses and functional aspects of the fluid reservoir 300 will be appreciated from the corresponding figures and the description above.

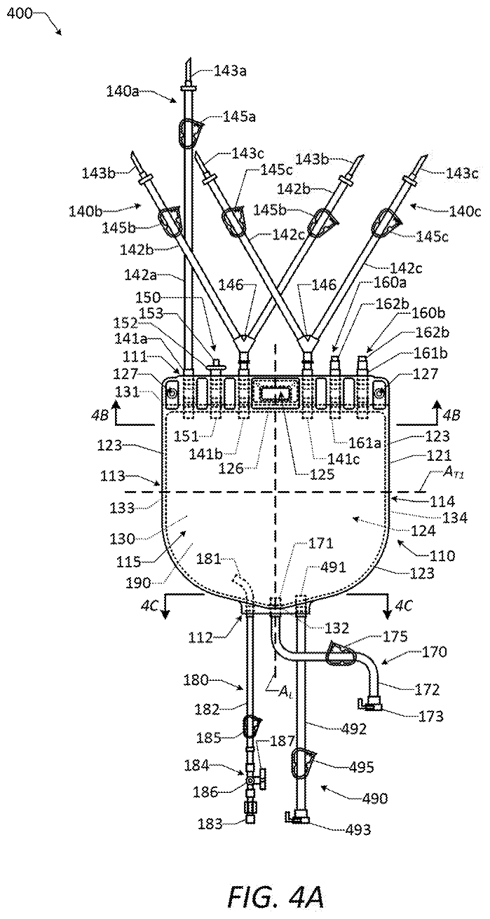

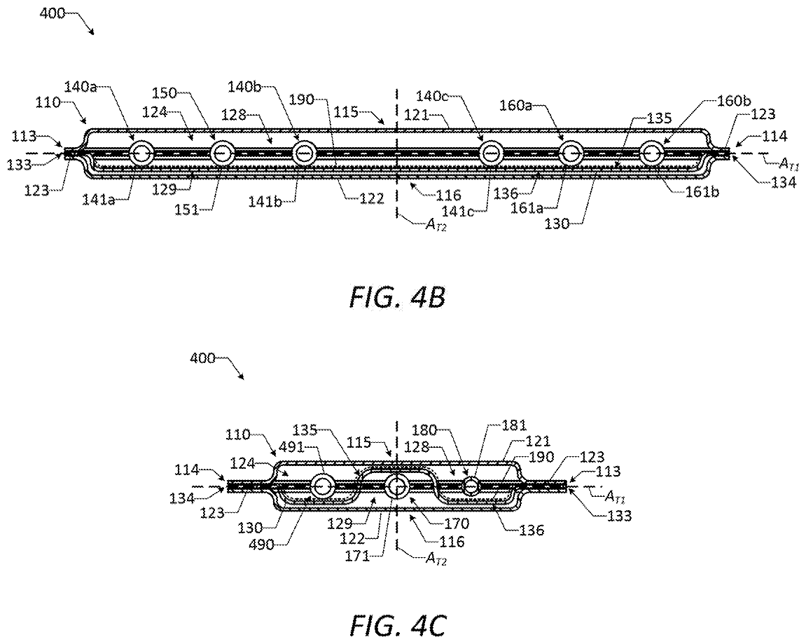

FIGS. 4A-4C illustrate a collapsible fluid reservoir 400 (which also may be referred to as a “medical fluid reservoir,” a “fluid reservoir” or simply a “reservoir”) and components thereof according to one or more embodiments of the disclosure. The collapsible fluid reservoir 400 may be configured for use in medical procedures to collect, store, and transfer biological fluids, such as blood. As shown, the collapsible fluid reservoir 400 generally may be configured in a manner similar to the collapsible fluid reservoir 100 described above, although certain differences are noted below. Like reference numbers indicate similar or identical components or features.

As shown, the collapsible fluid reservoir 400 may include the outflow line 170 configured in the manner described above. The fluid reservoir 400 also may include a second outflow line 490 (which also may be referred to as a “fluid outflow line,” an “exit line,” or an “outlet line”) attached to the container 110 and configured to allow biological fluid to exit the interior space 124 thereof. As shown, the second outflow line 490 may be disposed along the second end 112 of the container 110 and at least partially within the interior space 124. In this manner, a passage of the second outflow line 490 may be in fluid communication with the interior space 124 for receiving biological fluid therefrom. In various embodiments, the fluid reservoir 400 may include any number of outflow lines attached to the container 110 and configured to allow biological fluids to exit the interior space 124.

As shown in FIG. 4A, the second outflow line 490 may include a second outflow port 491, a second outflow tube 492, a second outflow fitting 493, and a second outflow clamp 495. As shown, at least a portion of the second outflow line 490 may be disposed within the interior space 124, and at least a portion of the second outflow line 490 may be disposed outside of the interior space 124. In particular, at least a portion of the second outflow port 491, including a first end thereof, may be disposed within the interior space 124, and at least a portion of the second outflow port 491, including an opposite second end thereof, and the second outflow tube 492 may be disposed outside of the interior space 124 and extend away from the container 110. As shown in FIG. 4C, the portion of the second outflow port 491 disposed within the interior space 124 may be disposed within the first portion 128 thereof. In other words, the portion of the second outflow port 491 disposed within the interior space 124 may be disposed between the third side 135 of the filter 130 and the first sheet 121 of the container 110. In this manner, the second outflow line 490 may be in fluid communication with the first portion 129 of the interior space 124 and configured to receive biological fluid therefrom, upstream of the filter 130. The second outflow port 491 may be fixedly attached to the first sheet 121 and/or the second sheet 122, for example, by RF welding, heat sealing, one or more adhesives, or other means of attachment. A first end of the second outflow tube 492 may be fixedly attached to the second end of the second outflow port 491, and the second outflow tube 492 may extend away from the container 110. An opposite second end of the second outflow tube 492 may have the second outflow fitting 493 fixedly attached thereto. The second outflow fitting 493 may be configured for attaching to other components of an extracorporeal circuit. In certain embodiments, as shown, the second outflow fitting 493 may be formed as a lock fitting for releasably locking onto another component, although other types of fittings may be used. The second outflow clamp 495 may be disposed about and positioned around a portion of the second outflow tube 492 near the second outflow fitting 493. The second outflow clamp 495 may be an adjustable clamp configured to be selectively moved between an open position for allowing fluid flow through the second outflow tube 492 and a closed position for preventing fluid flow through the second outflow tube 492.

In certain embodiments, the fluid reservoir 400 may be assembled by sealing or welding components of the reservoir 400 to one another. In certain embodiments, the assembly process may begin by positioning the first sheet 121, the second sheet 122, the support member 126, the filter 130, the inflow ports 141a, 141b, 141c, the vent port 151, the auxiliary ports 161a, 161b, the outflow ports 171, 491, and the return port 181 as shown in FIGS. 4A-4C. The seal 123 then may be formed, for example, by heat sealing or RF welding, along the respective outer peripheries of the first sheet 121, the second sheet 122, and the filter 130. In this manner, the first sheet 121, the second sheet 122, the filter 130, the inflow ports 141a, 141b, 141c, the vent port 151, the auxiliary ports 161a, 162a, the outflow ports 171, 491, and the return port 181 may be fixedly attached to one another by the seal 123. In certain embodiments, the first sheet 121 and the second sheet 122 may be directly attached to one another along one or more first portions of the seal 123, and the first sheet 121 and the second sheet 122 may be indirectly attached to one another along one or more second portions of the seal 123. For example, according to the illustrated embodiment, the first sheet 121 and the second sheet 122 may be directly attached to one another along respective first portions of the seal 123 positioned along the first end 111 of the container 110. However, the first sheet 121 and the second sheet 122 may be indirectly attached to one another via other components of the fluid reservoir 400 along respective second portions of the seal 123 where the other components are positioned between the first sheet 121 and the second sheet 122. As shown, the first sheet 121 and the second sheet 122 may be indirectly attached to one another via the filter 130 along respective second portions of the seal 123 positioned along the periphery of the filter 130. Additionally, the first sheet 121 and the second sheet 122 may be indirectly attached to one another via the support member 126, the filter 130, the inflow ports 141a, 141b, 141c, the vent port 151, the auxiliary ports 161a, 161b, the outflow ports 171, 491, and/or the return port 181 along respective second portions of the seal 123 where respective portions of such components are positioned between the first sheet 121 and the second sheet 122, as shown. In certain embodiments, the remaining components of the inflow lines 140a, 140b, 140c, the vent line 150, the auxiliary lines 160a, 160b, the outflow lines 170, 490, and the return line 180 may be attached prior to forming the seal 123. In other embodiments, such components may be attached after forming the seal 123.

During use, the fluid reservoir 400 may be oriented in a vertical manner, as shown in FIG. 4A, with the reservoir 400 suspended by a mating structure engaging the central opening 125 and/or the lateral openings 127 of the container 110. Biological fluid may be delivered into the interior space 124 of the container 110 via one or more of the inflow lines 140. In particular, unfiltered biological fluid may be delivered through one or more of the inflow lines 140 and into the first portion 128 of the interior space 124. The biological fluid may pass from the first portion 128, through the filter 130, and into the second portion 129 of the interior space 124, as the filter 130 removes undesired materials or substances from the biological fluid. The filtered biological fluid may pass from the second portion 129 of the interior space 124 and through the outflow line 170 for use in a medical procedure. Additionally or alternatively, the unfiltered biological fluid may pass from the first portion 128 of the interior space 124 and through the second outflow line 490 for use in a medical procedure or other purposes. The outflow line 170 and the second outflow line 490 may be used simultaneously or at different times to draw desired amounts of the filtered and unfiltered biological fluid from the interior space 124 of the container 110, as may vary during a medical procedure and between different medical procedures. Other uses and functional aspects of the fluid reservoir 400 will be appreciated from the corresponding figures and the description above.

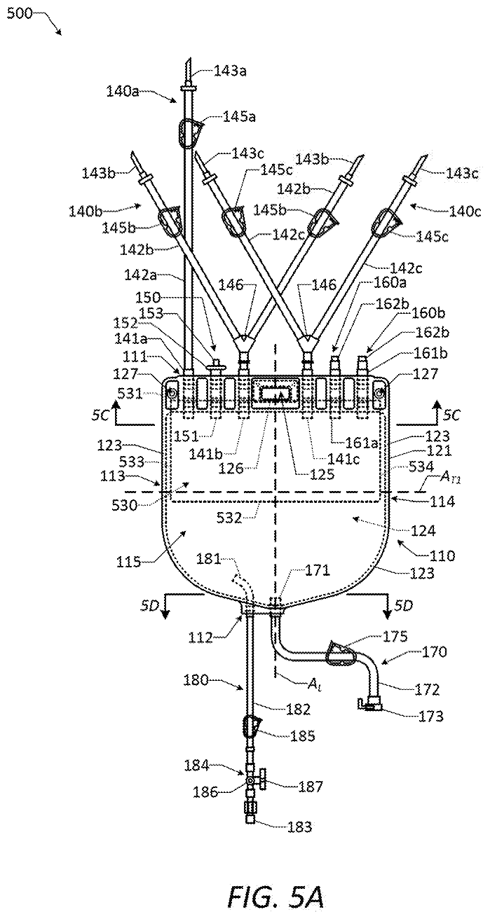

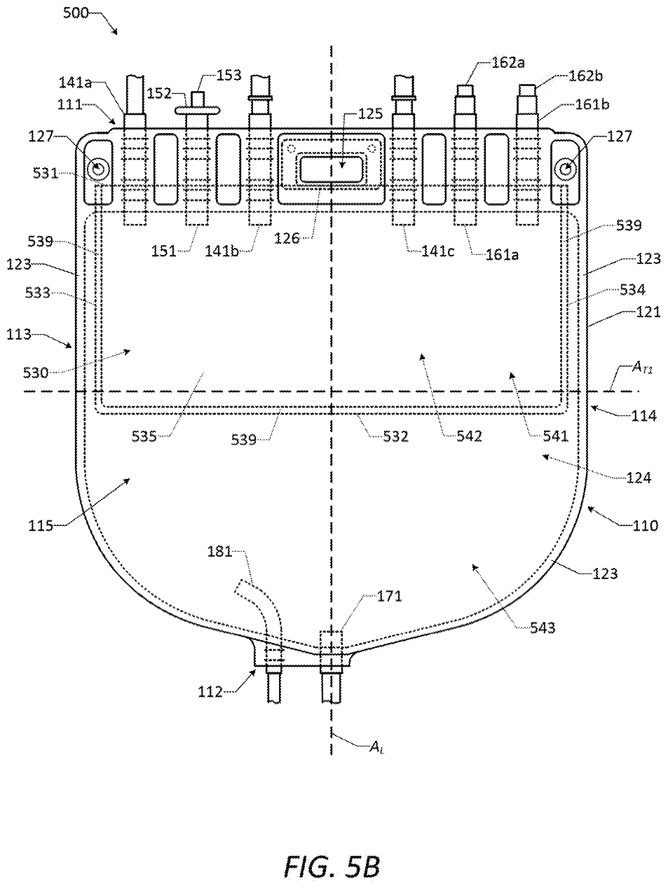

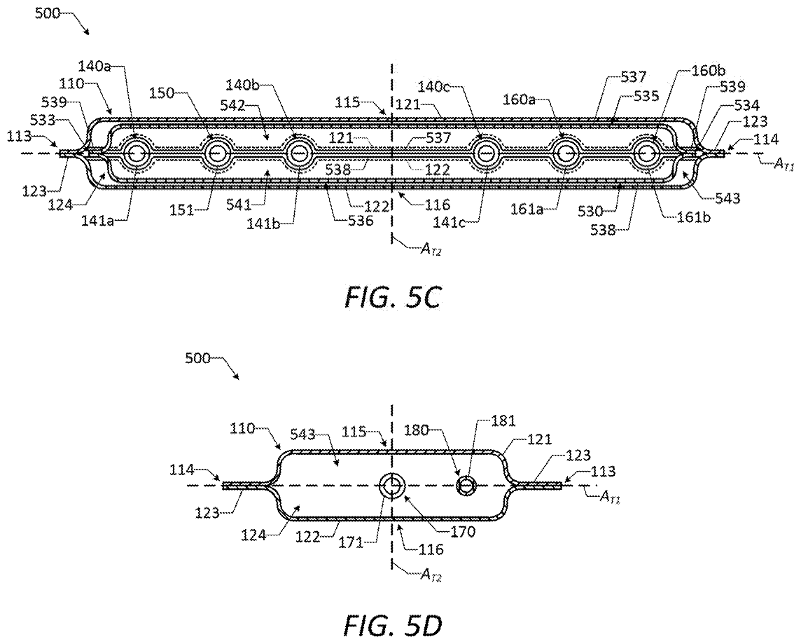

FIGS. 5A-5D illustrate a collapsible fluid reservoir 500 (which also may be referred to as a “medical fluid reservoir,” a “fluid reservoir” or simply a “reservoir”) and components thereof according to one or more embodiments of the disclosure. The collapsible fluid reservoir 500 may be configured for use in medical procedures to collect, store, and transfer biological fluids, such as blood. As shown, the collapsible fluid reservoir 500 generally may be configured in a manner similar to the collapsible fluid reservoir 100 described above, although certain differences are noted below. Like reference numbers indicate similar or identical components or features.

As shown, the collapsible fluid reservoir 500 may include a flexible filter 530 instead of the flexible filter 130 described above. The flexible filter 530 (which also may be referred to as a “filter bag,” a “filter member,” a “filter assembly,” or a “filter”) may be configured to remove one or more materials or substances from a biological fluid during use of the fluid reservoir 500. For example, the fluid reservoir 500 may be configured for use with blood, and the filter 530 may be configured to remove clots and small clumps of platelets and white blood cells that may form during collection and storage of the blood. Alternatively, the fluid reservoir 500 may be used with other types of biological materials, and the filter 530 may be configured to remove undesired materials from the biological material collected therein. The filter 530 may have an elongated, substantially planar shape, as shown, although the filter 530 may be flexed or deformed into a variety of different shapes. As shown, the filter 530 may have a first end 531 (which also may be referred to as a “top end” or an “open end”) and a second end 532 (which also may be referred to as a “bottom end” or a “closed end”) positioned opposite one another in the direction of the longitudinal axis A_{L} of the container 110, a first side 533 (which also may be referred to as a “first lateral side”) and a second side 534 (which also may be referred to as a “second lateral side”) positioned opposite one another in the direction of the first transverse axis A_{T1} of the container 110, and a third side 535 (which also may be referred to as a “front side”) and a fourth side 536 (which also may be referred to as a “back side”) positioned opposite one another in the direction of the second transverse axis A_{T2} of the container 110. The fluid reservoir 500 may be configured for use in a vertical orientation, as shown in FIG. 5A, with the first end 531 positioned above the second end 532 during use of the reservoir 500.

In certain embodiments, as shown, the filter 530 may be formed as a bag of filter material that is open along the first end 531 thereof and closed (aside from pores of the filter material) along the second end 532 and the sides 533, 534, 535, 536 thereof. In certain embodiments, the filter 530 may be formed of a plastic, although other suitable materials may be used. The filter 530 may include a plurality of pores defined therein and configured to allow the biological material to pass therethrough while removing the undesired materials. According to various embodiments, the filter 530 may have a pore size between 1 micron and 1000 microns, between 1 micron and 500 microns, between 200 and 250 microns, or about 230 microns. In certain embodiments, the filter 530 may have a pore size that is constant or substantially constant throughout the filter 530. For example, the filter 530 may have a pore size that is constant in the direction of the longitudinal axis A_{L} of the container 110 and in the direction of the first transverse axis A_{T1} of the container 110. In certain embodiments, the filter 530 may have a pore size that varies throughout the filter 530. For example, the filter 530 may have a pore size that decreases in a direction from the first end 531 to the second end 532 of the filter 530 over at least a portion of the length of the filter 530. As another example, the filter 530 may have a pore size that increases in a direction from the first end 531 to the second end 532 of the filter 530 over at least a portion of the length of the filter 530. In certain embodiments, the filter 530 may include two or more regions each having a different, constant or substantially constant pore size throughout the respective region.

In certain embodiments, as shown, the filter 530 may include a pair of flexible sheets attached to one another. In particular, the filter 530 may include a first sheet 537 (which also may be referred to as a “first filter sheet”) and a second sheet 538 (which also may be referred to as a “second filter sheet”) formed of a filter material and attached to one another. As shown, the sheets 537, 538 may be attached to one another along the second end 532, the first side 533, and the second side 534 of the filter 530 and may be unattached to one another along the first end 531 and along an interior region inward of the periphery of the filter 530. In certain embodiments, the sheets 537, 538 may be fixedly attached to one another by a seal 539 (which also may be referred to as a “filter seal”) formed along the second end 532, the first side 533, and the second side 534 of the filter 530, for example, by RF welding, heat sealing, one or more adhesives, or other means of attachment. As shown, the first sheet 537 and the second sheet 538 may define an interior space 541 therebetween for receiving a biological fluid. Each of the sheets 537, 538 may have an elongated, substantially planar shape, although the sheets 537, 538 may be flexed or deformed into a variety of different shapes. In certain embodiments, as shown, each of the sheets 537, 538 may extend along the entire length of the filter 530, along the entire width of the filter 530, and along the entire periphery of the filter 530. In certain embodiments, the first sheet 537 may extend along the third side 535 of the filter 530, and the second sheet 538 may extend along the fourth side 536 of the filter 530. In certain embodiments, each of the sheets 537, 538 may have a rectangular or substantially rectangular shape, as shown, although other shapes may be used. In other embodiments, the filter 530 may be formed from a single sheet of filter material that is folded along the second end 532 of the filter 530, sealed along the first side 533 by a first seal 539, and sealed along the second side 534 by a second seal 539. In still other embodiments, the filter 530 may be formed as a unitary structure of filter material having a bag configuration without any seals.

As shown in FIGS. 5A-5C, the filter 530 may be disposed at least partially within the interior space 124 of the container 110. In certain embodiments, as shown, the filter 530 may be attached to the container 110 along a first portion of the outer periphery of the filter 530 and unattached to the container 110 along a second portion of the outer periphery of the filter 530. For example, a first portion of the filter 530 extending along the first end 531 of the filter 530 may be fixedly and directly attached to at least one of the first sheet 121 and the second sheet 122 of the container 110, while a second portion of the filter 530 may be unattached to, and configured to move relative to, the container 110. In particular, the first portion of the filter 530 may be fixedly and directly attached to the container 110 by the seal 123 thereof, which may be formed by RF welding, heat sealing, one or more adhesives, or other means of attachment. In certain embodiments, as shown, the width of the filter 530 may be less than the width of the interior space 124 of the container 110, and the length of the filter 530 may be less than the length of the interior space 124 of the container 110. In this manner, the second end 532, the first side 533, and the second side 534 may be offset inwardly from the respective portions of the seal 123 of the container 110, and the volume of the interior space 541 of the filter 530 may be less than the volume of the interior space 124 of the container 110.

As shown, the filter 530 may divide the interior space 124 of the container 110 into two separate portions, with the filter 530 extending therebetween. In particular, a first portion 542 (which also may be referred to as an “inflow portion” or an “upstream portion”) of the interior space 124 may be defined between the interior surfaces of the filter 530 and a first portion of the seal 123 of the container 110, and a second portion 543 (which also may be referred to as an “outflow portion” or a “downstream portion”) of the interior space 124 may be defined between the exterior surfaces of the filter 530 and a second portion of the seal 123 of the container 110. In other words, the interior space 541 of the filter 530 may be the first portion 542 of the interior space 124 of the container 110, and the remainder of the interior space 124 may be the second portion 543 of the interior space 124 of the container 110. In this manner, biological fluid may be delivered into the first portion 542, and the filter 530 may remove undesired materials therefrom as the biological fluid passes through the filter 530 from the first portion 542 into the second portion 543 of the interior space 124. As described above, the first sheet 121, the second sheet 122, and the filter 530 may be flexible, and thus these components may flex or deform, respectively, to accommodate a first volume of the biological fluid in the first portion 542 and a second volume of the filtered biological fluid in the second portion 543. Additional features and functionality of the filter 530 will be appreciated from the corresponding figures and the description of the fluid reservoir 500 provided herein.

As shown, the respective passages of the inflow lines 140a, 140b, 140c each may be in fluid communication with the first portion 542 of the interior space 124. In particular, respective portions of the inflow ports 141a, 141b, 141c, including the first ends thereof, may be positioned within the first portion 542 of the interior space 124 (i.e., within the interior space 541 of the filter 530) for delivering the biological fluid thereto, upstream of the filter 530. In a similar manner, the passage of the vent line 150 also may be in fluid communication with the first portion 542 of the interior space 124. In particular, a portion of the vent port 151, including the first end thereof, may be positioned within the first portion 542 of the interior space 124 (i.e., within the interior space 541 of the filter 530) for receiving air or other gases therefrom. Further, the respective passages of the auxiliary lines 160a, 160b each may be in fluid communication with the first portion 542 of the interior space 124. In particular, respective portions of the auxiliary ports 161a, 161b, including the first ends thereof, may be positioned within the first portion 542 of the interior space 124 (i.e., within the interior space 541 of the filter 530) for delivering additional fluids thereto, upstream of the filter 530.

In certain embodiments, the fluid reservoir 500 may be assembled by sealing or welding components of the reservoir 500 to one another. In certain embodiments, the assembly process may begin by positioning the first sheet 537 and the second sheet 538 as shown in FIGS. 5A-5C. The seal 539 of the filter 530 then may be formed, for example, by heat sealing or RF welding, along the second end 532, the first side 533, and the second side 534 thereof. Next, the first sheet 121, the second sheet 122, the support member 126, the filter 530, the inflow ports 141a, 141b, 141c, the vent port 151, the auxiliary ports 161a, 161b, the outflow port 171, and the return port 181 may be positioned as shown in FIGS. 5A-5D. The seal 123 then may be formed, for example, by heat sealing or RF welding, along the respective outer peripheries of the first sheet 121 and the second sheet 122. In this manner, the first sheet 121, the second sheet 122, the filter 530, the inflow ports 141a, 141b, 141c, the vent port 151, the auxiliary ports 161a, 161b, the outflow port 171, and the return port 181 may be fixedly attached to one another by the seal 123. In certain embodiments, the first sheet 121 and the second sheet 122 may be directly attached to one another along one or more first portions of the seal 123, and the first sheet 121 and the second sheet 122 may be indirectly attached to one another along one or more second portions of the seal 123. For example, according to the illustrated embodiment, the first sheet 121 and the second sheet 122 may be directly attached to one another along respective first portions of the seal 123 positioned along the first end 111, the second end 112, the first side 113, and the second side 114 of the container 110. However, the first sheet 121 and the second sheet 122 may be indirectly attached to one another via other components of the fluid reservoir 500 along respective second portions of the seal 123 where the other components are positioned between the first sheet 121 and the second sheet 122. As shown, the first sheet 121 and the second sheet 122 may be indirectly attached to one another via the filter 530 along respective second portions of the seal 123 positioned along the first portion of the filter 530. Additionally, the first sheet 121 and the second sheet 122 may be indirectly attached to one another via the support member 126, the filter 530, the inflow ports 141a, 141b, 141c, the vent port 151, the auxiliary ports 161a, 161b, the outflow port 171, and/or the return port 181 along respective second portions of the seal 123 where respective portions of such components are positioned between the first sheet 121 and the second sheet 122, as shown. In certain embodiments, the remaining components of the inflow lines 140a, 140b, 140c, the vent line 150, the auxiliary lines 160a, 160b, the outflow line 170, and the return line 180 may be attached prior to forming the seal 123. In other embodiments, such components may be attached after forming the seal 123.

During use, the fluid reservoir 500 may be oriented in a vertical manner, as shown in FIG. 5A, with the reservoir 500 suspended by a mating structure engaging the central opening 125 and/or the lateral openings 127 of the container 110. Biological fluid may be delivered into the interior space 124 of the container 110 via one or more of the inflow lines 140. In particular, unfiltered biological fluid may be delivered through one or more of the inflow lines 140 and into the first portion 542 of the interior space 124. The biological fluid may pass from the first portion 542, through the filter 530, and into the second portion 543 of the interior space 124, as the filter 530 removes undesired materials or substances from the biological fluid. The filtered biological fluid may pass from the second portion 543 of the interior space 124 and through the outflow line 170 for use in a medical procedure. Meanwhile, air or other gases may exit the interior space 124 through the vent line 150. In particular, air or other gases may pass from the first portion 542 of the interior space 124 and through the vent line 150, while the biological fluid is prevented from passing through the vent line 150 by the vent filter 152 thereof. Additional fluids may be delivered into the interior space 124 via one or more of the auxiliary lines 160. In particular, additional fluids may be delivered through one or more of the auxiliary lines 160 and into the first portion 542 of the interior space 124. Other uses and functional aspects of the fluid reservoir 500 will be appreciated from the corresponding figures and the description above.

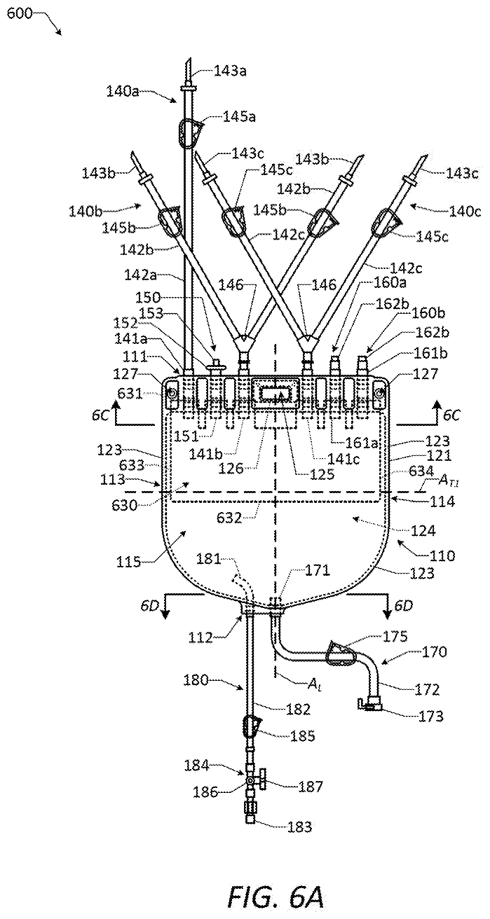

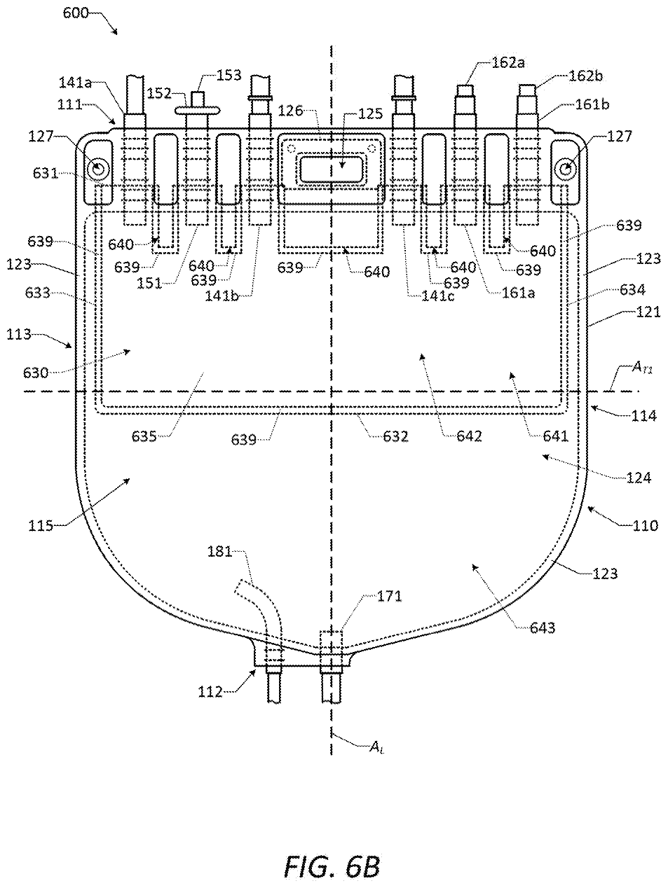

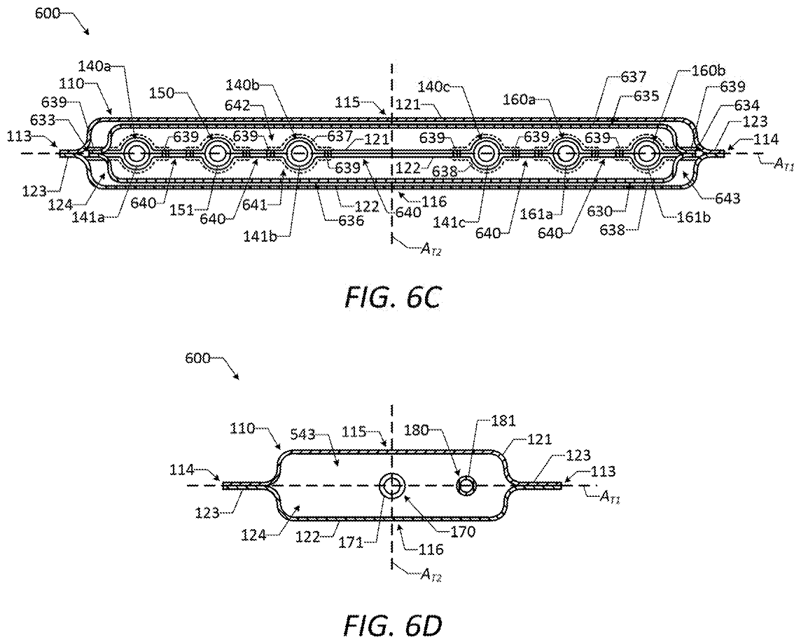

FIGS. 6A-6D illustrate a collapsible fluid reservoir 600 (which also may be referred to as a “medical fluid reservoir,” a “fluid reservoir” or simply a “reservoir”) and components thereof according to one or more embodiments of the disclosure. The collapsible fluid reservoir 600 may be configured for use in medical procedures to collect, store, and transfer biological fluids, such as blood. As shown, the collapsible fluid reservoir 600 generally may be configured in a manner similar to the collapsible fluid reservoir 500 described above, although certain differences are noted below. Like reference numbers indicate similar or identical components or features.

As shown, the collapsible fluid reservoir 600 may include a flexible filter 630 instead of the flexible filter 530 described above. The flexible filter 630 (which also may be referred to as a “filter bag,” a “filter member,” a “filter assembly,” or a “filter”) may be configured to remove one or more materials or substances from a biological fluid during use of the fluid reservoir 600. For example, the fluid reservoir 600 may be configured for use with blood, and the filter 630 may be configured to remove clots and small clumps of platelets and white blood cells that may form during collection and storage of the blood. Alternatively, the fluid reservoir 600 may be used with other types of biological materials, and the filter 630 may be configured to remove undesired materials from the biological material collected therein. The filter 630 may have an elongated, substantially planar shape, as shown, although the filter 630 may be flexed or deformed into a variety of different shapes. As shown, the filter 630 may have a first end 631 (which also may be referred to as a “top end” or an “open end”) and a second end 632 (which also may be referred to as a “bottom end” or a “closed end”) positioned opposite one another in the direction of the longitudinal axis A_{L} of the container 110, a first side 633 (which also may be referred to as a “first lateral side”) and a second side 634 (which also may be referred to as a “second lateral side”) positioned opposite one another in the direction of the first transverse axis A_{T1} of the container 110, and a third side 635 (which also may be referred to as a “front side”) and a fourth side 636 (which also may be referred to as a “back side”) positioned opposite one another in the direction of the second transverse axis A_{T2} of the container 110. The fluid reservoir 600 may be configured for use in a vertical orientation, as shown in FIG. 6A, with the first end 631 positioned above the second end 632 during use of the reservoir 600.

In certain embodiments, as shown, the filter 630 may be formed as a bag of filter material that includes a plurality of openings along the first end 631 thereof and is closed (aside from pores of the filter material) along the second end 632 and the sides 633, 634, 635, 636 thereof. In certain embodiments, the filter 630 may be formed of a plastic, although other suitable materials may be used. The filter 630 may include a plurality of pores defined therein and configured to allow the biological material to pass therethrough while removing the undesired materials. According to various embodiments, the filter 630 may have a pore size between 1 micron and 1000 microns, between 1 micron and 500 microns, between 200 and 250 microns, or about 230 microns. In certain embodiments, the filter 630 may have a pore size that is constant or substantially constant throughout the filter 630. For example, the filter 630 may have a pore size that is constant in the direction of the longitudinal axis A_{L} of the container 110 and in the direction of the first transverse axis A_{T1} of the container 110. In certain embodiments, the filter 630 may have a pore size that varies throughout the filter 630. For example, the filter 630 may have a pore size that decreases in a direction from the first end 631 to the second end 632 of the filter 630 over at least a portion of the length of the filter 630. As another example, the filter 630 may have a pore size that increases in a direction from the first end 631 to the second end 632 of the filter 630 over at least a portion of the length of the filter 630. In certain embodiments, the filter 630 may include two or more regions each having a different, constant or substantially constant pore size throughout the respective region.

In certain embodiments, as shown, the filter 630 may include a pair of flexible sheets attached to one another. In particular, the filter 630 may include a first sheet 637 (which also may be referred to as a “first filter sheet”) and a second sheet 638 (which also may be referred to as a “second filter sheet”) formed of a filter material and attached to one another. As shown, the sheets 637, 638 may be attached to one another along the second end 632, the first side 633, the second side 634, and portions of the first end 631 of the filter 630 and may be unattached to one another at each of the openings along the first end 631 and along an interior region inward of the periphery of the filter 630. In certain embodiments, the sheets 637, 638 may be fixedly attached to one another by a plurality of seals 639 (which also may be referred to as “filter seals”) formed along the second end 632, the first side 633, and the second side 634 of the filter 630, and also along a plurality of U-shaped cutouts 640 extending from the first end 631 of the filter 630, for example, by RF welding, heat sealing, one or more adhesives, or other means of attachment. As shown, the first sheet 637 and the second sheet 638 may define an interior space 641 therebetween for receiving a biological fluid. Each of the sheets 637, 638 may have an elongated, substantially planar shape, although the sheets 637, 638 may be flexed or deformed into a variety of different shapes. In certain embodiments, as shown, each of the sheets 637, 638 may extend along the entire length of the filter 630, along the entire width of the filter 630, and along the entire periphery of the filter 630. In certain embodiments, the first sheet 637 may extend along the third side 635 of the filter 630, and the second sheet 638 may extend along the fourth side 636 of the filter 630. In certain embodiments, each of the sheets 637, 638 may have a rectangular or substantially rectangular shape, as shown, although other shapes may be used. In other embodiments, the filter 630 may be formed from a single sheet of filter material that is folded along the second end 632 of the filter 630, sealed along the first side 633 by a first seal 639, sealed along the second side 634 by a second seal 639, and sealed along the portions of the first end 631 by additional seals 639. In still other embodiments, the filter 630 may be formed as a unitary structure of filter material having a bag configuration without any seals.

As shown in FIGS. 6A-6C, the filter 630 may be disposed at least partially within the interior space 124 of the container 110. In certain embodiments, as shown, the filter 630 may be attached to the container 110 along a plurality of first portions of the outer periphery of the filter 630 and unattached to the container 110 along a plurality of second portions of the outer periphery of the filter 630. For example, a plurality of first portions of the filter 630 extending along the first end 631 of the filter 630 may be fixedly and directly attached to at least one of the first sheet 121 and the second sheet 122 of the container 110, while a plurality of second portions of the filter 630 may be unattached to, and configured to move relative to, the container 110. In particular, the first portions of the filter 630 may be fixedly and directly attached to the container 110 by the seal 123 thereof, which may be formed by RF welding, heat sealing, one or more adhesives, or other means of attachment. In certain embodiments, as shown, the width of the filter 630 may be less than the width of the interior space 124 of the container 110, and the length of the filter 630 may be less than the length of the interior space 124 of the container 110. In this manner, the second end 632, the first side 633, and the second side 634 may be offset inwardly from the respective portions of the seal 123 of the container 110, and the volume of the interior space 641 of the filter 630 may be less than the volume of the interior space 124 of the container 110.

As shown, the filter 630 may divide the interior space 124 of the container 110 into two separate portions, with the filter 630 extending therebetween. In particular, a first portion 642 (which also may be referred to as an “inflow portion” or an “upstream portion”) of the interior space 124 may be defined between the interior surfaces of the filter 630 and a plurality of first portions of the seal 123 of the container 110, and a second portion 643 (which also may be referred to as an “outflow portion” or a “downstream portion”) of the interior space 124 may be defined between the exterior surfaces of the filter 630 and a plurality of second portions of the seal 123 of the container 110. In other words, the interior space 641 of the filter 630 may be the first portion 642 of the interior space 124 of the container 110, and the remainder of the interior space 124 may be the second portion 643 of the interior space 124 of the container 110. In this manner, biological fluid may be delivered into the first portion 642, and the filter 630 may remove undesired materials therefrom as the biological fluid passes through the filter 630 from the first portion 642 into the second portion 643 of the interior space 124. As described above, the first sheet 121, the second sheet 122, and the filter 630 may be flexible, and thus these components may flex or deform, respectively, to accommodate a first volume of the biological fluid in the first portion 642 and a second volume of the filtered biological fluid in the second portion 643. Additional features and functionality of the filter 630 will be appreciated from the corresponding figures and the description of the fluid reservoir 600 provided herein.

As shown, the respective passages of the inflow lines 140a, 140b, 140c each may be in fluid communication with the first portion 642 of the interior space 124. In particular, respective portions of the inflow ports 141a, 141b, 141c, including the first ends thereof, may be positioned within the first portion 642 of the interior space 124 (i.e., within the interior space 641 of the filter 630) for delivering the biological fluid thereto, upstream of the filter 630. In a similar manner, the passage of the vent line 150 also may be in fluid communication with the first portion 642 of the interior space 124. In particular, a portion of the vent port 151, including the first end thereof, may be positioned within the first portion 642 of the interior space 124 (i.e., within the interior space 641 of the filter 630) for receiving air or other gases therefrom. Further, the respective passages of the auxiliary lines 160a, 160b each may be in fluid communication with the first portion 642 of the interior space 124. In particular, respective portions of the auxiliary ports 161a, 161b, including the first ends thereof, may be positioned within the first portion 642 of the interior space 124 (i.e., within the interior space 641 of the filter 630) for delivering additional fluids thereto, upstream of the filter 630.

In certain embodiments, the fluid reservoir 600 may be assembled by sealing or welding components of the reservoir 600 to one another. In certain embodiments, the assembly process may begin by positioning the first sheet 637 and the second sheet 638 as shown in FIGS. 6A-6C. The seals 639 of the filter 630 then may be formed, for example, by heat sealing or RF welding, along the second end 632, the first side 633, the second side 634, and each of the cutouts 640 thereof. Next, the first sheet 121, the second sheet 122, the support member 126, the filter 630, the inflow ports 141a, 141b, 141c, the vent port 151, the auxiliary ports 161a, 161b, the outflow port 171, and the return port 181 may be positioned as shown in FIGS. 6A-6D. The seal 123 then may be formed, for example, by heat sealing or RF welding, along the respective outer peripheries of the first sheet 121 and the second sheet 122. In this manner, the first sheet 121, the second sheet 122, the filter 630, the inflow ports 141a, 141b, 141c, the vent port 151, the auxiliary ports 161a, 161b, the outflow port 171, and the return port 181 may be fixedly attached to one another by the seal 123. It will be appreciated that the cutouts 640 of the filter 630 may ease formation of the seal 123. In particular, because the filter 630 is not present along the regions of the seal 123 spanning the cutouts 640, it may be easier to form the seal 123 by heat sealing or RF welding along these regions due to the reduced amount of material (i.e., fewer layers of material being sealed or welded). In certain embodiments, the first sheet 121 and the second sheet 122 may be directly attached to one another along one or more first portions of the seal 123, and the first sheet 121 and the second sheet 122 may be indirectly attached to one another along one or more second portions of the seal 123. For example, according to the illustrated embodiment, the first sheet 121 and the second sheet 122 may be directly attached to one another along respective first portions of the seal 123 positioned along the first end 111, the second end 112, the first side 113, and the second side 114 of the container 110. However, the first sheet 121 and the second sheet 122 may be indirectly attached to one another via other components of the fluid reservoir 600 along respective second portions of the seal 123 where the other components are positioned between the first sheet 121 and the second sheet 122. As shown, the first sheet 121 and the second sheet 122 may be indirectly attached to one another via the filter 630 along respective second portions of the seal 123 positioned along the first portions of the filter 630. Additionally, the first sheet 121 and the second sheet 122 may be indirectly attached to one another via the support member 126, the filter 630, the inflow ports 141a, 141b, 141c, the vent port 151, the auxiliary ports 161a, 161b, the outflow port 171, and/or the return port 181 along respective second portions of the seal 123 where respective portions of such components are positioned between the first sheet 121 and the second sheet 122, as shown. In certain embodiments, the remaining components of the inflow lines 140a, 140b, 140c, the vent line 150, the auxiliary lines 160a, 160b, the outflow line 170, and the return line 180 may be attached prior to forming the seal 123. In other embodiments, such components may be attached after forming the seal 123.

During use, the fluid reservoir 600 may be oriented in a vertical manner, as shown in FIG. 6A, with the reservoir 600 suspended by a mating structure engaging the central opening 125 and/or the lateral openings 127 of the container 110. Biological fluid may be delivered into the interior space 124 of the container 110 via one or more of the inflow lines 140. In particular, unfiltered biological fluid may be delivered through one or more of the inflow lines 140 and into the first portion 642 of the interior space 124. The biological fluid may pass from the first portion 642, through the filter 630, and into the second portion 643 of the interior space 124, as the filter 630 removes undesired materials or substances from the biological fluid. The filtered biological fluid may pass from the second portion 643 of the interior space 124 and through the outflow line 170 for use in a medical procedure. Meanwhile, air or other gases may exit the interior space 124 through the vent line 150. In particular, air or other gases may pass from the first portion 642 of the interior space 124 and through the vent line 150, while the biological fluid is prevented from passing through the vent line 150 by the vent filter 152 thereof. Additional fluids may be delivered into the interior space 124 via one or more of the auxiliary lines 160. In particular, additional fluids may be delivered through one or more of the auxiliary lines 160 and into the first portion 642 of the interior space 124. Other uses and functional aspects of the fluid reservoir 600 will be appreciated from the corresponding figures and the description above.

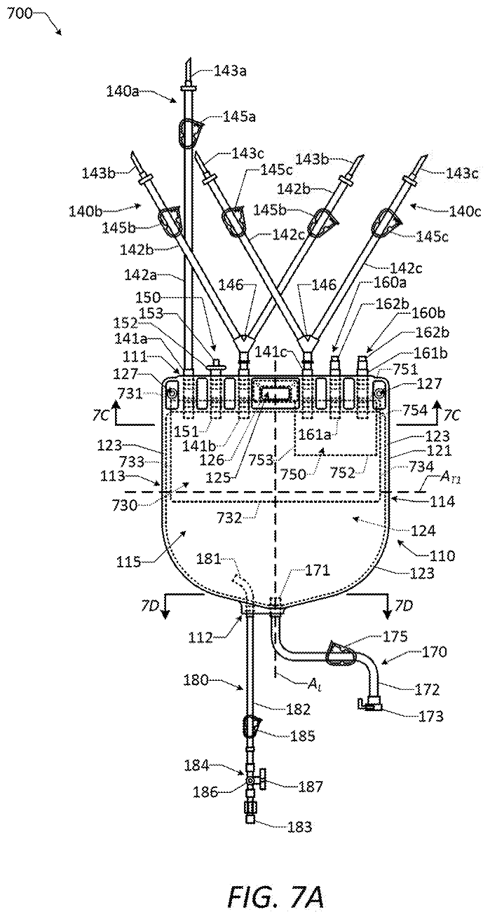

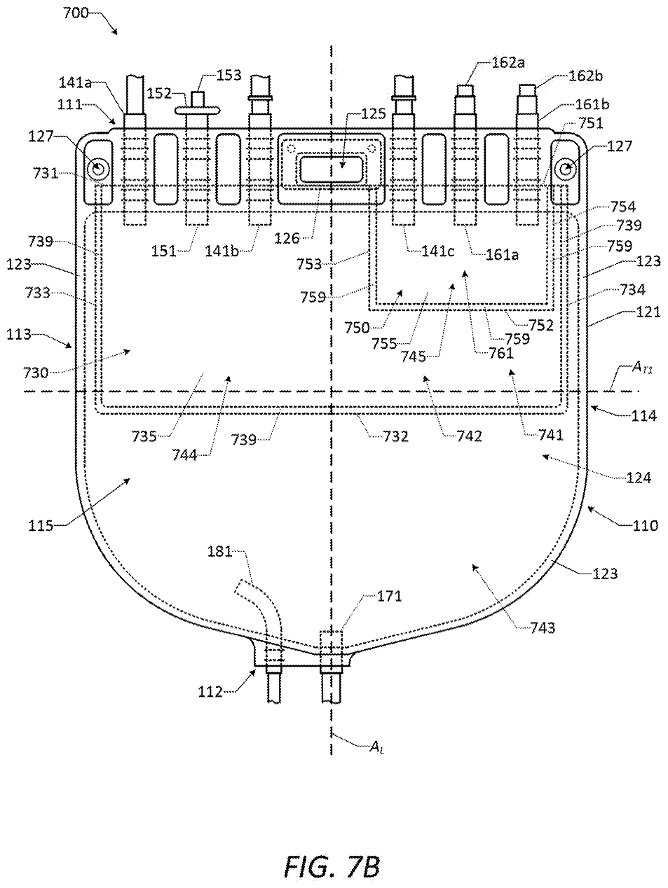

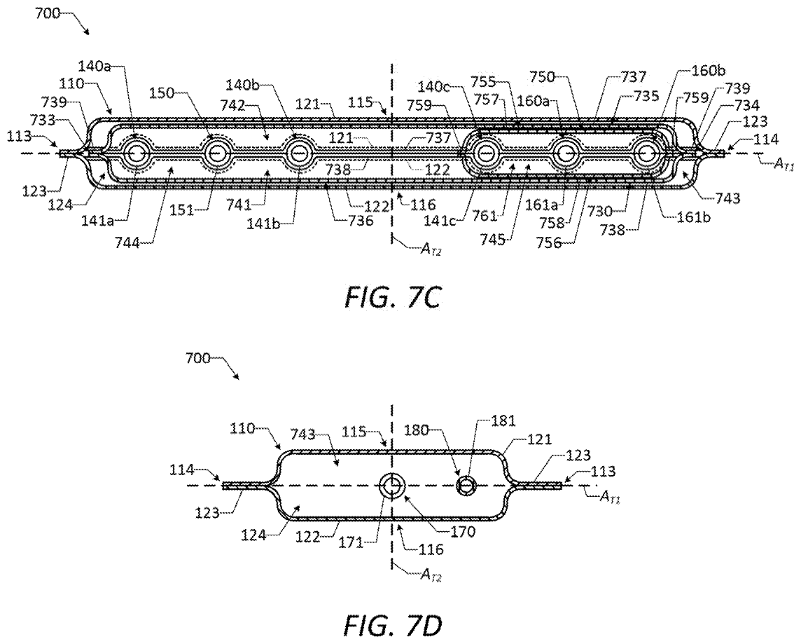

FIGS. 7A-7D illustrate a collapsible fluid reservoir 700 (which also may be referred to as a “medical fluid reservoir,” a “fluid reservoir” or simply a “reservoir”) and components thereof according to one or more embodiments of the disclosure. The collapsible fluid reservoir 700 may be configured for use in medical procedures to collect, store, and transfer biological fluids, such as blood. As shown, the collapsible fluid reservoir 700 generally may be configured in a manner similar to the collapsible fluid reservoir 500 described above, although certain differences are noted below. Like reference numbers indicate similar or identical components or features.

As shown, the collapsible fluid reservoir 700 may include a plurality of flexible filters. In particular, the collapsible fluid reservoir 700 may include a first flexible filter 730 and a second flexible filter 750 positioned at least partially within the first flexible filter 730. The first flexible filter 730 (which also may be referred to as a “first filter bag,” an “outer filter bag,” a “first filter member,” a “first filter assembly,” or a “first filter”) and the second flexible filter 750 (which also may be referred to as a “second filter bag,” an “inner filter bag,” a “second filter member,” a “second filter assembly,” or a “second filter”) each may be configured to remove one or more materials or substances from a biological fluid during use of the fluid reservoir 700. For example, the fluid reservoir 700 may be configured for use with blood, and the filters 730, 750 may be configured to remove clots and small clumps of platelets and white blood cells that may form during collection and storage of the blood. Alternatively, the fluid reservoir 700 may be used with other types of biological materials, and the filters 730, 750 may be configured to remove undesired materials from the biological material collected therein. The filters 730, 750 may have an elongated, substantially planar shape, as shown, although the filters 730, 750 may be flexed or deformed into a variety of different shapes. As shown, the first filter 730 may have a first end 731 (which also may be referred to as a “top end” or an “open end”) and a second end 732 (which also may be referred to as a “bottom end” or a “closed end”) positioned opposite one another in the direction of the longitudinal axis A_{L} of the container 110, a first side 733 (which also may be referred to as a “first lateral side”) and a second side 734 (which also may be referred to as a “second lateral side”) positioned opposite one another in the direction of the first transverse axis A_{T1} of the container 110, and a third side 735 (which also may be referred to as a “front side”) and a fourth side 736 (which also may be referred to as a “back side”) positioned opposite one another in the direction of the second transverse axis A_{T2} of the container 110. The fluid reservoir 700 may be configured for use in a vertical orientation, as shown in FIG. 7A, with the first end 731 positioned above the second end 732 during use of the reservoir 700.

In certain embodiments, as shown, the first filter 730 may be formed as a bag of filter material that is open along the first end 731 thereof and closed (aside from pores of the filter material) along the second end 732 and the sides 733, 734, 735, 736 thereof. In certain embodiments, the first filter 730 may be formed of a plastic, although other suitable materials may be used. The first filter 730 may include a plurality of pores defined therein and configured to allow the biological material to pass therethrough while removing the undesired materials. According to various embodiments, the first filter 730 may have a pore size between 1 micron and 1000 microns, between 1 micron and 500 microns, between 200 and 250 microns, or about 230 microns. In certain embodiments, the first filter 730 may have a pore size that is constant or substantially constant throughout the first filter 730. For example, the first filter 730 may have a pore size that is constant in the direction of the longitudinal axis A_{L} of the container 110 and in the direction of the first transverse axis A_{T1} of the container 110. In certain embodiments, the first filter 730 may have a pore size that varies throughout the first filter 730. For example, the first filter 730 may have a pore size that decreases in a direction from the first end 731 to the second end 732 of the first filter 730 over at least a portion of the length of the first filter 730. As another example, the first filter 730 may have a pore size that increases in a direction from the first end 731 to the second end 732 of the first filter 730 over at least a portion of the length of the first filter 730. In certain embodiments, the first filter 730 may include two or more regions each having a different, constant or substantially constant pore size throughout the respective region.

In certain embodiments, as shown, the first filter 730 may include a pair of flexible sheets attached to one another. In particular, the may include a first sheet 737 (which also may be referred to as a “first outer filter sheet”) and a second sheet 738 (which also may be referred to as a “second outer filter sheet”) formed of a filter material and attached to one another. As shown, the sheets 737, 738 may be attached to one another along the second end 732, the first side 733, and the second side 734 of the first filter 730 and may be unattached to one another along the first end 731 and along an interior region inward of the periphery of the first filter 730. In certain embodiments, the sheets 737, 738 may be fixedly attached to one another by a seal 739 (which also may be referred to as an “outer filter seal”) formed along the second end 732, the first side 733, and the second side 734 of the first filter 730, for example, by RF welding, heat sealing, one or more adhesives, or other means of attachment. As shown, the first sheet 737 and the second sheet 738 may define an interior space 741 therebetween for receiving a biological fluid. Each of the sheets 737, 738 may have an elongated, substantially planar shape, although the sheets 737, 738 may be flexed or deformed into a variety of different shapes. In certain embodiments, as shown, each of the sheets 737, 738 may extend along the entire length of the first filter 730, along the entire width of the first filter 730, and along the entire periphery of the first filter 730. In certain embodiments, the first sheet 737 may extend along the third side 735 of the first filter 730, and the second sheet 738 may extend along the fourth side 736 of the first filter 730. In certain embodiments, each of the sheets 737, 738 may have a rectangular or substantially rectangular shape, as shown, although other shapes may be used. In other embodiments, the first filter 730 may be formed from a single sheet of filter material that is folded along the second end 732 of the first filter 730, sealed along the first side 733 by a first seal 739, and sealed along the second side 734 by a second seal 739. In still other embodiments, the first filter 730 may be formed as a unitary structure of filter material having a bag configuration without any seals.

As shown in FIGS. 7A-7C, the first filter 730 may be disposed at least partially within the interior space 124 of the container 110. In certain embodiments, as shown, the first filter 730 may be attached to the container 110 along a first portion of the outer periphery of the first filter 730 and unattached to the container 110 along a second portion of the outer periphery of the first filter 730. For example, a first portion of the first filter 730 extending along the first end 731 of the first filter 730 may be fixedly and directly attached to at least one of the first sheet 121 and the second sheet 122 of the container 110, while a second portion of the first filter 730 may be unattached to, and configured to move relative to, the container 110. In particular, the first portion of the first filter 730 may be fixedly and directly attached to the container 110 by the seal 123 thereof, which may be formed by RF welding, heat sealing, one or more adhesives, or other means of attachment. In certain embodiments, as shown, the width of the first filter 730 may be less than the width of the interior space 124 of the container 110, and the length of the first filter 730 may be less than the length of the interior space 124 of the container 110. In this manner, the second end 732, the first side 733, and the second side 734 may be offset inwardly from the respective portions of the seal 123 of the container 110, and the volume of the interior space 741 of the first filter 730 may be less than the volume of the interior space 124 of the container 110.

As shown, the first filter 730 may divide the interior space 124 of the container 110 into two separate portions, with the first filter 730 extending therebetween. In particular, a first portion 742 (which also may be referred to as an “inflow portion” or an “upstream portion”) of the interior space 124 may be defined between the interior surfaces of the first filter 730 and a first portion of the seal 123 of the container 110, and a second portion 743 (which also may be referred to as an “outflow portion” or a “downstream portion”) of the interior space 124 may be defined between the exterior surfaces of the first filter 730 and a second portion of the seal 123 of the container 110. In other words, the interior space 741 of the first filter 730 may be the first portion 742 of the interior space 124 of the container 110, and the remainder of the interior space 124 may be the second portion 743 of the interior space 124 of the container 110. In this manner, biological fluid may be delivered into the first portion 742, and the first filter 730 may remove undesired materials therefrom as the biological fluid passes through the first filter 730 from the first portion 742 into the second portion 743 of the interior space 124. As described above, the first sheet 121, the second sheet 122, and the first filter 730 may be flexible, and thus these components may flex or deform, respectively, to accommodate a first volume of the biological fluid in the first portion 742 and a second volume of the filtered biological fluid in the second portion 743. Additional features and functionality of the first filter 730 will be appreciated from the corresponding figures and the description of the fluid reservoir 700 provided herein.

In a similar manner, the second filter 750 may have a first end 751 (which also may be referred to as a “top end” or an “open end”) and a second end 752 (which also may be referred to as a “bottom end” or a “closed end”) positioned opposite one another in the direction of the longitudinal axis A_{L} of the container 110, a first side 753 (which also may be referred to as a “first lateral side”) and a second side 754 (which also may be referred to as a “second lateral side”) positioned opposite one another in the direction of the first transverse axis A_{T1} of the container 110, and a third side 755 (which also may be referred to as a “front side”) and a fourth side 756 (which also may be referred to as a “back side”) positioned opposite one another in the direction of the second transverse axis A_{T2} of the container 110. The fluid reservoir 700 may be configured for use in a vertical orientation, as shown in FIG. 7A, with the first end 751 positioned above the second end 752 during use of the reservoir 700.

In certain embodiments, as shown, the second filter 750 may be formed as a bag of filter material that is open along the first end 751 thereof and closed (aside from the pores of the filter material) along the second end 752 and the sides 753, 754, 755, 756 thereof. In certain embodiments, the second filter 750 may be formed of a plastic, although other suitable materials may be used. The second filter 750 may include a plurality of pores defined therein and configured to allow the biological material to pass therethrough while removing the undesired materials. According to various embodiments, the second filter 750 may have a pore size between 1 micron and 1000 microns, between 1 micron and 500 microns, between 200 and 250 microns, or about 230 microns. In certain embodiments, the second filter 750 may have a pore size that is greater than a pore size of the first filter 730. In certain embodiments, the second filter 750 may have a pore size that is constant or substantially constant throughout the second filter 750. For example, the second filter 750 may have a pore size that is constant in the direction of the longitudinal axis A_{L} of the container 110 and in the direction of the first transverse axis A_{T1} of the container 110. In certain embodiments, the second filter 750 may have a pore size that varies throughout the second filter 750. For example, the second filter 750 may have a pore size that decreases in a direction from the first end 751 to the second end 752 of the second filter 750 over at least a portion of the length of the second filter 750. As another example, the second filter 750 may have a pore size that increases in a direction from the first end 751 to the second end 752 of the second filter 750 over at least a portion of the length of the second filter 750. In certain embodiments, the second filter 750 may include two or more regions each having a different, constant or substantially constant pore size throughout the respective region.

In certain embodiments, as shown, the second filter 750 may include a pair of flexible sheets attached to one another. In particular, the may include a first sheet 757 (which also may be referred to as a “first inner filter sheet”) and a second sheet 758 (which also may be referred to as a “second inner filter sheet”) formed of a filter material and attached to one another. As shown, the sheets 757, 758 may be attached to one another along the second end 752, the first side 753, and the second side 754 of the second filter 750 and may be unattached to one another along the first end 751 and along an interior region inward of the periphery of the second filter 750. In certain embodiments, the sheets 757, 758 may be fixedly attached to one another by a seal 759 (which also may be referred to as an “inner filter seal”) formed along the second end 752, the first side 753, and the second side 754 of the second filter 750, for example, by RF welding, heat sealing, one or more adhesives, or other means of attachment. As shown, the first sheet 757 and the second sheet 758 may define an interior space 761 therebetween for receiving a biological fluid. Each of the sheets 757, 758 may have an elongated, substantially planar shape, although the sheets 757, 758 may be flexed or deformed into a variety of different shapes. In certain embodiments, as shown, each of the sheets 757, 758 may extend along the entire length of the second filter 750, along the entire width of the second filter 750, and along the entire periphery of the second filter 750. In certain embodiments, the first sheet 757 may extend along the third side 755 of the second filter 750, and the second sheet 758 may extend along the fourth side 756 of the second filter 750. In certain embodiments, each of the sheets 757, 758 may have a rectangular or substantially rectangular shape, as shown, although other shapes may be used. In other embodiments, the second filter 750 may be formed from a single sheet of filter material that is folded along the second end 752 of the second filter 750, sealed along the first side 753 by a first seal 759, and sealed along the second side 754 by a second seal 759. In still other embodiments, the second filter 750 may be formed as a unitary structure of filter material having a bag configuration without any seals.

As shown in FIGS. 7A-7C, the second filter 750 may be disposed at least partially within the interior space 124 of the container 110 and at least partially within the interior space 741 of the first filter 730. In certain embodiments, as shown, the second filter 750 may be attached to the container 110 and the first filter 730 along a first portion of the outer periphery of the second filter 750 and unattached to the container 110 and the first filter 730 along a second portion of the outer periphery of the second filter 750. For example, a first portion of the second filter 750 extending along the first end 751 of the second filter 750 may be fixedly and directly attached to at least one of the first sheet 121 and the second sheet 122 of the container 110 or the first sheet 737 and the second sheet 738 of the first filter 730, while a second portion of the second filter 750 may be unattached to, and configured to move relative to, the container 110 and the first filter 730. In certain embodiments, the first portion of the second filter 750 may be fixedly attached to the container 110 by the seal 123 thereof, which may be formed by RF welding, heat sealing, one or more adhesives, or other means of attachment. In certain embodiments, as shown, the width of the second filter 750 may be less than the width of the interior space 124 of the container 110 and less than the width of the interior space 741 of the first filter 730, and the length of the second filter 750 may be less than the length of the interior space 124 of the container 110 and less than the length of the interior space 741 of the first filter 730. In this manner, the second end 752, the first side 753, and the second side 754 may be offset inwardly from the respective portions of the seal 123 of the container 110 and the respective portions of the seal 739 of the first filter 730, and the volume of the interior space 761 of the second filter 750 may be less than the volume of the interior space 124 of the container 110 and less than the volume of the interior space 741 of the first filter 730.

As shown, the second filter 750 may divide the interior space 741 of the first filter 730 into two separate portions, with the second filter 750 extending therebetween. In particular, a first portion 744 of the interior space 741 may be defined between the interior surfaces of the second filter 750 and a first portion of the seal 123 of the container 110, and a second portion 745 of the interior space 741 may be defined between the exterior surfaces of the second filter 750, a second portion of the seal 123 of the container 110, and the interior surfaces of the first seal 730. In other words, the interior space 761 of the second filter 750 may be the first portion 744 of the interior space 741 of the first filter 730, and the remainder of the interior space 741 may be the second portion 745 of the interior space 741 of the first filter 730. In this manner, biological fluid may be delivered into the first portion 744, and the second filter 750 may remove undesired materials therefrom as the biological fluid passes through the second filter 750 from the first portion 744 into the second portion 745 of the interior space 741. As described above, the first filter 730 and the second filter 750 may be flexible, and thus these components may flex or deform, respectively to accommodate a first volume of the biological fluid in the first portion 744 and a second volume of the filtered biological fluid in the second portion 745. Additional features and functionality of the second filter 750 will be appreciated from the corresponding figures and the description of the fluid reservoir 700 provided herein.

As shown, the respective passages of the inflow lines 140a, 140b, 140c each may be in fluid communication with the first portion 742 of the interior space 124. In particular, respective portions of the inflow ports 141a, 141b, 141c, including the first ends thereof, may be positioned within the first portion 742 of the interior space 124 (i.e., within the interior space 741 of the first filter 730) for delivering the biological fluid thereto, upstream of the first filter 730. As shown, a respective portion of the inflow port 141c, including the first end thereof, may be positioned within the first portion 744 of the interior space 741 (i.e., within the interior space 761 of the second filter 750). In a similar manner, the passage of the vent line 150 also may be in fluid communication with the first portion 742 of the interior space 124. In particular, a portion of the vent port 151, including the first end thereof, may be positioned within the first portion 742 of the interior space 124 (i.e., within the interior space 741 of the first filter 730) for receiving air or other gases therefrom. Further, the respective passages of the auxiliary lines 160a, 160b each may be in fluid communication with the first portion 742 of the interior space 124. In particular, respective portions of the auxiliary ports 161a, 161b, including the first ends thereof, may be positioned within the first portion 742 of the interior space 124 (i.e., within the interior space 741 of the first filter 730) for delivering additional fluids thereto, upstream of the first filter 730. Additionally, respective portions of the auxiliary ports 161a, 161b, including the first ends thereof, may be positioned within the first portion 744 of the interior space 741 (i.e., within the interior space 761 of the second filter 750).

In certain embodiments, the fluid reservoir 700 may be assembled by sealing or welding components of the reservoir 700 to one another. In certain embodiments, the assembly process may begin by positioning the first sheet 737 and the second sheet 738 as shown in FIGS. 7A-7C. The seal 739 of the first filter 730 then may be formed, for example, by heat sealing or RF welding, along the second end 732, the first side 733, and the second side 734 thereof. In a similar manner, the first sheet 757 and the second sheet 758 may be positioned as shown in FIGS. 7A-7C, and the seal 759 of the second filter 750 then may be formed, for example, by heat sealing or RF welding, along the second end 752, the first side 753, and the second side 754 thereof. Next, the first sheet 121, the second sheet 122, the support member 126, the first filter 730, the second filter 750, the inflow ports 141a, 141b, 141c, the vent port 151, the auxiliary ports 161a, 161b, the outflow port 171, and the return port 181 may be positioned as shown in FIGS. 7A-7D. The seal 123 then may be formed, for example, by heat sealing or RF welding, along the respective outer peripheries of the first sheet 121 and the second sheet 122. In this manner, the first sheet 121, the second sheet 122, the first filter 730, the second filter 750, the inflow ports 141a, 141b, 141c, the vent port 151, the auxiliary ports 161a, 161b, the outflow port 171, and the return port 181 may be fixedly attached to one another by the seal 123. In certain embodiments, the first sheet 121 and the second sheet 122 may be directly attached to one another along one or more first portions of the seal 123, and the first sheet 121 and the second sheet 122 may be indirectly attached to one another along one or more second portions of the seal 123. For example, according to the illustrated embodiment, the first sheet 121 and the second sheet 122 may be directly attached to one another along respective first portions of the seal 123 positioned along the first end 111, the second end 112, the first side 113, and the second side 114 of the container 110. However, the first sheet 121 and the second sheet 122 may be indirectly attached to one another via other components of the fluid reservoir 700 along respective second portions of the seal 123 where the other components are positioned between the first sheet 121 and the second sheet 122. As shown, the first sheet 121 and the second sheet 122 may be indirectly attached to one another via the first filter 730 and the second filter 750 along respective second portions of the seal 123 positioned along the first portions of the first filter 730 and the second filter 750. Additionally, the first sheet 121 and the second sheet 122 may be indirectly attached to one another via the support member 126, first filter 730, the second filter 750, the inflow ports 141a, 141b, 141c, the vent port 151, the auxiliary ports 161a, 161b, the outflow port 171, and/or the return port 181 along respective second portions of the seal 123 where respective portions of such components are positioned between the first sheet 121 and the second sheet 122, as shown. In certain embodiments, the remaining components of the inflow lines 140a, 140b, 140c, the vent line 150, the auxiliary lines 160a, 160b, the outflow line 170, and the return line 180 may be attached prior to forming the seal 123. In other embodiments, such components may be attached after forming the seal 123.

During use, the fluid reservoir 700 may be oriented in a vertical manner, as shown in FIG. 7A, with the reservoir 700 suspended by a mating structure engaging the central opening 125 and/or the lateral openings 127 of the container 110. Biological fluid may be delivered into the interior space 124 of the container 110 via one or more of the inflow lines 140. In particular, unfiltered biological fluid may be delivered through one or more of the inflow lines 140 and into the first portion 742 of the interior space 124. For example, biological fluid may be delivered into the first portion 744 of the interior space 741 of the first filter 730 via the inflow lines 140a, 140b. Such biological fluid may pass from the first portion 742 (i.e., the first portion 744 of the interior space 741 of the first filter 730), through the first filter 730, and into the second portion 743 of the interior space 124, as the first filter 730 removes undesired materials or substances from the biological fluid. Such filtered biological fluid then may pass from the second portion 743 of the interior space 124 and through the outflow line 170 for use in a medical procedure. As another example, biological fluid may be delivered into the second portion 745 of the interior space 741 of the first filter 730 (i.e., the interior space 761 of the second filter 750) via the inflow line 140c. Such biological fluid may pass from the interior space 761 of the second filter 750, through the second filter 750, and into the first portion 744 of the interior space 741 of the first filter 730, as the second filter 750 removes undesired materials or substances from the biological fluid. Such filtered biological fluid then may pass from the first portion 744 of the interior space 741 of the first filter 730, through the first filter 730, and into the second portion 743 of the interior space 124, as the first filter 730 further removes undesired materials or substances from the biological fluid. Such filtered biological fluid then may pass from the second portion 743 of the interior space 124 and through the outflow line 170 for use in a medical procedure. As described above, in certain embodiments, the second filter 750 may have a pore size that is greater than a pore size of the first filter 730. In this manner, biological fluid that is delivered into the interior space 761 of the second filter 750 via the inflow line 140c may be filtered first by the second filter 750 to remove larger materials or substances and then filtered again by the first filter 730 to remove smaller materials or substances. Meanwhile, air or other gases may exit the interior space 124 through the vent line 150. In particular, air or other gases may pass from the first portion 742 of the interior space 124 and through the vent line 150, while the biological fluid is prevented from passing through the vent line 150 by the vent filter 152 thereof. Additional fluids may be delivered into the interior space 124 via one or more of the auxiliary lines 160. In particular, additional fluids may be delivered through one or more of the auxiliary lines 160 and into the first portion 642 of the interior space 124, in particular, the interior space 761 of the second filter 750. Other uses and functional aspects of the fluid reservoir 600 will be appreciated from the corresponding figures and the description above.

Many modifications of the embodiments of the present disclosure will come to mind to one skilled in the art to which the disclosure pertains upon having the benefit of the teachings presented herein through the foregoing descriptions and the associated drawings. Therefore, it is to be understood that the present invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A collapsible fluid reservoir comprising:
a flexible container comprising a first sheet and a second sheet attached to one another and defining an internal space therebetween for containing a biological fluid;
a flexible filter disposed within the internal space and attached to the container along an entire periphery of the filter, the filter comprising a first side facing the first sheet and a second side opposite the first side and facing the second sheet;
a first inflow line disposed at least partially within the interior space and between the first side of the filter and the first sheet;
a second inflow line disposed at least partially within the interior space and between the first side of the filter and the first sheet; and
an outflow line disposed at least partially within the interior space and between the second side of the filter and the second sheet.

2. The collapsible fluid reservoir of claim 1, wherein the first sheet, the second sheet, and the filter are attached to one another by a seal extending along the periphery of the filter.

3. The collapsible fluid reservoir of claim 1, wherein the first sheet and the second sheet are formed of polyvinyl chloride, and wherein the filter comprises a sheet of filter material.

4. The collapsible fluid reservoir of claim 1, wherein the filter has a pore size between 1 micron and 1000 microns.

5. The collapsible fluid reservoir of claim 1, wherein the filter has a pore size that is substantially constant throughout the filter.

6. The collapsible fluid reservoir of claim 1, wherein the filter has a pore size that varies throughout the filter.

7. The collapsible fluid reservoir of claim 1, wherein the first inflow line and the second inflow line are disposed along a first end of the container, wherein the outflow line is disposed along an opposite second end of the container, and wherein the collapsible fluid reservoir is configured for use in a vertical orientation with the first end of the container disposed above the second end of the container.

8. The collapsible fluid reservoir of claim 1, wherein the first inflow line comprises a first inflow port disposed at least partially within the interior space and a first inflow tube attached to the first inflow port and disposed at least partially outside of the interior space, wherein the second inflow line comprises a second inflow port disposed at least partially within the interior space and a second inflow tube attached to the second inflow port and disposed at least partially outside of the interior space, and wherein the outflow line comprises an outflow port disposed at least partially within the interior space and an outflow tube attached to the outflow port and disposed at least partially outside of the interior space.

9. The collapsible fluid reservoir of claim 1, further comprising a third inflow line disposed at least partially within the interior space and between the second side of the filter and the second sheet.

10. The collapsible fluid reservoir of claim 1, further comprising a return line disposed at least partially within the interior space and between the first side of the filter and the first sheet, wherein the first inflow line and the second inflow line are disposed along a first end of the container, wherein the outflow line and the return line are disposed along an opposite second end of the container, and wherein the collapsible fluid reservoir is configured for use in a vertical orientation with the first end of the container disposed above the second end of the container.

11. The collapsible fluid reservoir of claim 1, further comprising a vent line disposed at least partially within the interior space and between the first side of the filter and the first sheet, wherein the first inflow line, the second inflow line, and the vent line are disposed along a first end of the container, wherein the outflow line is disposed along an opposite second end of the container, and wherein the collapsible fluid reservoir is configured for use in a vertical orientation with the first end of the container disposed above the second end of the container.

12. The collapsible fluid reservoir of claim 1, further comprising a defoaming agent to facilitate a reduction of bubbles in the biological fluid.

13. A collapsible fluid reservoir comprising:
a flexible container comprising a first sheet and a second sheet attached to one another and defining an internal space therebetween for containing a biological fluid;
a flexible filter disposed within the internal space, the filter comprising a first side facing the first sheet and a second side opposite the first side and facing the second sheet;
a first inflow line disposed at least partially within the interior space and between the first side of the filter and the first sheet;
a second inflow line disposed at least partially within the interior space and between the second side of the filter and the second sheet; and
an outflow line disposed at least partially within the interior space and between the second side of the filter and the second sheet.

14. The collapsible fluid reservoir of claim 13, wherein the first inflow line and the second inflow line are disposed along a first end of the container, wherein the outflow line is disposed along an opposite second end of the container, and wherein the collapsible fluid reservoir is configured for use in a vertical orientation with the first end of the container disposed above the second end of the container.

15. A collapsible fluid reservoir comprising:
a flexible container comprising a first sheet and a second sheet attached to one another and defining an internal space therebetween for containing a biological fluid;
a flexible filter disposed within the internal space, the filter comprising a first side facing the first sheet and a second side opposite the first side and facing the second sheet;
an inflow line disposed at least partially within the interior space and between the first side of the filter and the first sheet;
a first outflow line disposed at least partially within the interior space and between the second side of the filter and the second sheet; and
a second outflow line disposed at least partially within the interior space and between the first side of the filter and the first sheet.

16. The collapsible fluid reservoir of claim 15, wherein the inflow line is disposed along a first end of the container, wherein the first outflow line and the second outflow line are disposed along an opposite second end of the container, and wherein the collapsible fluid reservoir is configured for use in a vertical orientation with the first end of the container disposed above the second end of the container.

17. A collapsible fluid reservoir comprising:
a flexible container comprising a first container sheet and a second container sheet attached to one another and defining a first internal space therebetween for containing a biological fluid;
a flexible filter bag attached to the container and disposed at least partially within the first internal space, the filter bag having a first side facing the first container sheet and a second side disposed opposite the first side and facing the second container sheet, and the filter bag defining a second internal space therein;
an inflow line disposed at least partially within the second internal space and at least partially within the first internal space; and
an outflow line disposed outside of the second internal space and at least partially within the first internal space.

18. The collapsible fluid reservoir of claim 17, wherein the first container sheet and the second container sheet are attached to one another by a container seal extending along the outer periphery of the container, and wherein the filter bag is attached to the container by the container seal.

19. The collapsible fluid reservoir of claim 18, wherein the filter bag has a first end, a second end disposed opposite the first end, a third side, and a fourth side disposed opposite the third side, wherein the filter bag is attached to the container by the container seal along the first end of the filter bag, and wherein the filter bag is unattached to the container along the second end, the first side, the second side, the third side, and the fourth side of the filter bag.

20. The collapsible fluid reservoir of claim 19, wherein the filter bag comprises a first filter sheet and a second filter sheet attached to one another and defining the second internal space therebetween.

21. The collapsible fluid reservoir of claim 20, wherein the first filter sheet and the second filter sheet are attached to one another by a filter seal extending along the second end, the third side, and the fourth side of the filter bag.

22. The collapsible fluid reservoir of claim 18, further comprising a flexible second filter bag attached to the container and disposed at least partially within the second internal space and at least partially within the first internal space, the second filter bag defining a third interior space therein.

23. The collapsible fluid reservoir of claim 22, wherein the second filter bag has a first end and a second end disposed opposite the first end, wherein the second filter bag is attached to the container by the container seal along the first end of the second filter bag, and wherein the second filter bag is unattached to the container along the second end of the filter bag.

24. The collapsible fluid reservoir of claim 22, wherein the second filter bag comprises a first filter sheet and a second filter sheet attached to one another by a filter seal and defining the third internal space therebetween.

25. The collapsible fluid reservoir of claim 22, wherein a pore size of the second filter bag is less than a pore size of the first filter bag.

26. The collapsible fluid reservoir of claim 22, wherein a volume of the third interior space is less than a volume of the second interior space.

27. The collapsible fluid reservoir of claim 22, wherein the inflow line is disposed outside of the third internal space.

28. The collapsible fluid reservoir of claim 27, further comprising a second inflow line disposed at least partially within the third internal space, at least partially within the second internal space, and at least partially within the first internal space.

29. The collapsible fluid reservoir of claim 17, further comprising:
a second inflow line disposed at least partially within the second internal space and at least partially within the first internal space;
a vent line disposed at least partially within the second internal space and at least partially within the first internal space; and
a return line disposed outside of the second internal space and at least partially within the first internal space.

30. The collapsible fluid reservoir of claim 17, wherein the inflow line is disposed along a first end of the container, wherein the outflow line is disposed along an opposite second end of the container, and wherein the collapsible fluid reservoir is configured for use in a vertical orientation with the first end of the container disposed above the second end of the container.
